(19) United States
(12) Patent Application Publication
(10) Pub. No.: US 2006/0084095 A1
(43) Pub. Date: Apr. 20, 2006

(54) Compositions, methods, and systems for determining bovine parentage and identity

(75) Inventors: David Rosenfeld, Sacramento, CA (US); Richard Kerr, Davis, CA (US); Sue DeNise, Davis, CA (US)

Correspondence Address: DLA PIPER RUDNICK GRAY CARY US, LLP, 4365 EXECUTIVE DRIVE, SUITE 1100, SAN DIEGO, CA, 92121-2133, US

(73) Assignee: MMI Genomics, Inc., Davis, CA (US)

(21) Appl. No.: 11/222,296

(22) Filed: Sep. 7, 2005

Related U.S. Application Data
(60) Provisional application No. 60/608,313, filed on Sep. 8, 2004.

(51) Int. Cl. C12Q 1/68

(52) U.S. Cl. 435/6

(57) ABSTRACT

Provided herein are methods to discover and use single nucleotide polymorphisms (SNP) for identifying parentage or identity of a bovine subject. The present invention further provides specific nucleic acid sequences, SNPs, and SNP patterns that can be used for identifying parentage of various breeds of cattle including Angus, Holstein, Limousin, Brahman, Hereford, Simmental, Gelbvieh, Charolais and Beefmaster breeds.

### RELATED APPLICATION

This application relies for priority under 35 U.S.C. 119(e) on U.S. provisional application No. 60/608,313, filed Sep. 8, 2004, the content of which is incorporated herein by refrence in its entirety.

### FIELD OF THE INVENTION

The invention relates generally to genetic markers and more specifically to polymorphisms associated with bovine parentage and identity.

### BACKGROUND INFORMATION

The analysis of identity and parentage is an important aspect of livestock evaluation. Classification of individual animals in a livestock population has often relied on a priori groupings of individual animals on the basis of parentage and registration with a Breed Association. Two possible options for classifying an individual animal, such as a bovine animal, into a population are:
1) Assign an animal to a population based on known or assumed parentage, phenotypic appearance or trait value for some phenotype, or
2) From a set of predefined populations, sample DNA from a number of members of each population to estimate allele frequencies in each population. Using the allele frequencies, it is possible to compute the likelihood a given genotype originated in each population and individuals can be assigned to population on the basis of these likelihoods (Pritchard, J. K., et al., Genetics 155: 945-959 (2000)).

DNA analysis provides a powerful tool for verifying the parentage and identification of individual animals. Microsatellite marker panels have been developed for cattle (Sherman et al., Anim Genet. 35(3):220-6.; Heyen et al., Anim Genet.28(l):21-27) and canine (See e.g., U.S. Pat. No. 5,874,217.; Ostrander et al., Mammalian Genome, 6: 192-195; Franscisco et al., Mammalian Genome 7:359-362) that are highly polymorphic and amenable to standardization among laboratories performing these tests. However, microsatellite scoring requires considerable human oversight and microsatellite markers have high mutation rates. Single nucleotide polymorphisms (SNP) are likely to become the standard marker for parentage verification and identity because of the ease of scoring, low cost assay development and high-throughput capability. There have been limited studies to evaluate the usefulness of SNP markers in small populations of animals (Heaton et al., Mamm Genome. 13(5):272-81; Werner et al., Anim. Genet. 35(l):44-9).

Compared with other types of DNA markers, single nucleotide polymorphisms (SNPs) are attractive because they are abundant, genetically stable, and amenable to high-throughput automated analysis. In cattle, the challenge has been to identify a minimal set of SNPs with sufficient power for use in a variety of popular breeds and crossbred populations. SNPs are DNA sequence variations that occur when a single nucleotide in the animal mt-DNA or nuclear genome sequence is altered and detected by traditionally direct DNA sequencing protocol. For example, a SNP might change the DNA sequence AAGGCTAA to ATGGCTAA. SNPs occur at one SNP every 1.9 kilobases in the human genome. SNPs can occur in both coding (gene) and noncoding regions of the genome. Many SNPs have no effect on cell function, but it is believed that others could predispose organism to disease or influence their response to a challenge. SNPs are evolutionarily stable—not changing much from generation to generation—making them easier to follow in population studies. SNPs also have properties that make them particularly attractive for genetic studies. They are more frequent than microsatellite markers, providing markers near to or in the locus of interest, some located within the gene (cSNP), which can directly influence protein structure or expression levels, giving insights into functional mechanisms.

Accordingly, there remains a need for methods and compositions that provide information regarding bovine parentage and identity.

### SUMMARY OF THE INVENTION

The present invention is based, in part, on the discovery of bovine single nucleotide polymorphism (SNP) markers that are associated with, and predictive of, bovine parentage and identity. Accordingly, the present invention provides methods to discover and use single nucleotide polymorphisms (SNP) for identifying parentage or identity of a bovine subject. The present invention further provides specific nucleic acid sequences, SNPs, and SNP patterns that can be used for identifying parentage for all bovine breeds, including but not limited to Angus, Limousin, Brahman, Hereford, Simmental, Gelbvieh, Charolais and Beefmaster breeds.

Accordingly, in one embodiment the present invention provides a method to infer parentage of a bovine subject from a nucleic acid sample of the bovine subject, that includes identifying in the nucleic acid sample, at least one nucleotide occurrence of at least one single nucleotide polymorphism (SNP) corresponding to the first nucleotide in the 3′ position to any one of SEQ ID NOs:261-390, wherein the SNP is associated with partentage, thereby inferring the identity of the bovine subject. A SNP is associated with parentage when at least one nucleotide occurrence of the SNP occurs more frequently in subjects of a particular lineage of animals than other lineages in a statistically significant manner, for example with greater than 80%, 85%, 90%, 95%, or 99% confidence. Therefore, in certain aspects, the methods include identifying whether the nucleotide occurrence is a bovine SNP allele identified herein as associated with bovine parengtage. The individual anilam can be any brred of cattle, including, but is not limited to, Angus, Limousin, Brahman, Simmental, Hereford, Gelbvieh or Charolais.

In another embodiment, the present invention provides a method for determining a nucleotide occurrence of a single nucleotide polymorphism (SNP) in a bovine sample, that includes contacting a bovine polynucleotide in the sample with an oligonucleotide that binds to a target region of any one of SEQ ID NOS:261 to 390 and determining the nucleotide occurrence of a single nucleotide polymorphism (SNP) corresponding to the first nucleotide in the 3′ position to any one of SEQ ID NOs:261-390, wherein the SNP is associated with partentage, thereby inferring the identity of the bovine subject. The determination typically includes analyzing binding of the oligonucleotide, or detecting an amplification product generated using the oligonucleotide, thereby determining the nucleotide occurrence of the SNP.

In another embodiment, the present invention provides a method to infer parentage of a bovine subject from a nucleic acid sample of the bovine subject, comprising identifying in the nucleic acid sample at least one nucleotide occurrence of at least one single nucleotide polymorphism (SNP) corresponding to the first nucleotide, or the complement thereof, in the 3′ position to any one of SEQ ID NOs:261-390, thereby inferring the identity of the bovine subject. The nucleotide incorporated immediately proximal to the 3′ end of each primer can be extendible or non-extendible nucleotide. In addition, the nucleotide can be fluorescently or chemically labeled. The target nucleic acid molecule can be DNA, RNA, single or double stranded.

In another embodiment, a method to infer parentage of a bovine subject from a nucleic acid sample of the bovine subject is provide. The method includes contacting the nucleic acid sample with a pair of oligonucleotides that comprise a primer pair, wherein amplified target nucleic acid molecules are produced; hybridizing at least one oligonucleotide primer selected from the group consisting of SEQ ID NOS:261-390 to one or more amplified target nucleic acid molecules, wherein each oligonucleotide primer is complementary to a specific and unique region of each target nucleic acid molecule such that the 3′ end of each primer is immediately proximal to a specific and unique target nucleotide of interest; extending each oligonucleotide with a template-dependent polymerase; and determining the identity of each nucleotide of interest by determining, for each extension primer employed, the identity of the nucleotide immediately proximal to the 3′ end of each primer. The primer pair can be any of the forward and reverse oligonucleotide primer pairs listed in Table 1. For example, a first primer of the primer pair can be selected from SEQ ID NOS: 1-130 and the second primer of the primer pair can be selected from SEQ ID NOS: 131-260.

In another embodiment, an isolated oligonucleotide comprising any one of SEQ ID NOS:261-390, is provided. Each oligonucleotide further includes one additional nucleotide positioned immediately proximal to the 3′ end of each oligonucleotide, wherein the oligonucleotide specifically hybridizes to a nucleic acid sequence derived from a bovine animal. Also provide is the complement of the aforementioned oligonucleotide.

In another embodiment, isolated oligonucleotide marker sets as set forth in Table 1 are provided.

In another embodiment, an isolated oligonucleotide marker set selected from from the group consisting of marker set MMIBP0001 through MMIBP0150 of Table.

In another embodiment, a method for identifying the parentage of a bovine test subject is provided. The method includes obtaining a nucleic acid sample from the test subject by a method comprising identifying in the nucleic acid sample at least one nucleotide occurrence of at least one single nucleotide polymorphism (SNP) corresponding to the first nucleotide, or the complement thereof, in the 3′ position to any one of SEQ ID NOs:261-390; and repeating the above for additional subjects; determining the allele frequency corresponding to each SNP identified; and comparing the allele frequency of the test subject with each additional subject. The additional bovine subjects can be the same breed or a different breed as the test subject.

In another embodiment, a kit for determining nucleotide occurrences of bovine SNPs is provided. Such a kit includes an oligonucleotide probe, primer, or primer pair, or combinations thereof, for identifying the nucleotide occurrence of at least one bovine single nucleotide polymorphism (SNP) corresponding to the first nucleotide, or the complement thereof, in the 3′ position to any one of SEQ ID NOs: 261-390, wherein the SNP is associated with parentage.

In another embodiment, a kit comprising at least one oligonucleotide marker set as set forth in Table 1, is provided. The marker set can be selected from the group consisting of marker set MMIBP0001 through MMIBP0150 of Table 1.

In another embodiment, a database including allele frequencies generated by identifying, in a nucleic acid sample derived from a bovine subject, the single nucleotide polymorphism (SNP) corresponding to the first nucleotide, or the complement thereof, in the 3′ position to each of the oligonucleotides set forth in SEQ ID NOS: 261-390, is provided.

In another embodiment, a database comprising allele frequencies generated by identifying, in a nucleic acid sample derived from a bovine subject, the single nucleotide polymorphisms (SNP) identified by the marker sets MMIBP0001 through MMIBP0150 of Table 1, is provided.

In yet another embodiment, a database comprising the allele frequencies set forth in Table 2, is provided.

In another embodiment, a computer-based method for identifying the parentage of a bovine subject, is provided. The method includes obtaining a nucleic acid sample from the bovine subject; identifying in the nucleic acid sample at least one nucleotide occurrence of at least one single nucleotide polymorphism (SNP) corresponding to the first nucleotide, or the complement thereof, in the 3′ position to any one of SEQ ID NOs: 261-390, searching a database comprising allele frequencies generated by the marker sets set forth in Table 1 or the allele frequencies set forth in Table 2; retrieving the information from database; optionally storing the information in a memory location associated with a user such that the information may be subsequently accessed and viewed by the user; and identifying the parentage of a bovine subject.

### DETAILED DESCRIPTION OF THE INVENTION

The present invention is based in part on the discovery of single nucleotide polymorphisms (SNPs) that can be used to infer parentage of a bovine subject. Accordingly, provided herein is a method for inferring the parentage of a bovine subject from a nucleic acid sample of the bovine subject, by identifying in the sample, a nucleotide occurrence for at least one single nucleotide polymorphism (SNP), wherein the nucleotide occurrence is associated with the parentage.

Using the teachings herein, SNPs associated with the parentage of any individual animal can be identified. Therefore, methods of the present invention for inferring parentage of a bovine subject, can be used to infer the parentage of any bovine subject regardless of breed. For example, the methods can be used to infer the parentage of an individual animal of a particular breed including, but not limited to, Angus, Limousin, Brahman, Simmental, Hereford, Holstein, Gelbvieh or Charolais cattle.

Since genomic DNA is double-stranded, each SNP can be defined in terms of either the plus strand or the minus strand. Thus, for every SNP, one strand will contain an immediately 5′-proximal invariant sequence and the other strand will contain an immediately 3′-distal invariant sequence. In one embodiment, an SNP of the present invention can be identified, in part, by its position immediately 3′ to any one of SEQ ID NOs: 261-390 in a target nucleic acid sequence. In another embodiment, an SNP of the invention can be identified as present in a nucleic acid sequence resulting from the replication of a nucleic acid sequence by any one of forward oligonucleotide primers SEQ ID NOS: 1-130 in combination with any one of reverse oligonucleotide primers SEQ ID NOS:131-260 (see e.g., Table 1, infra).

Nucleic acid molecules having a sequence complementary to that of an immediately 3′-distal invariant sequence of a SNP can, if extended in a “template-dependent” manner, form an extension product that would contain the SNP's polymorphic site. A preferred example of such a nucleic acid molecule is a nucleic acid molecule whose sequence is the same as that of a 5′-proximal invariant sequence of the SNP. “Template-dependent” extension refers to the capacity of a polymerase to mediate the extension of a primer such that the extended sequence is complementary to the sequence of a nucleic acid template. A “primer” is a single-stranded oligonucleotide (or oligonucleotide analog) or a single-stranded polynucleotide (or polynucleotide analog) that is capable of being extended by the covalent addition of a nucleotide (or nucleotide analog) in a “template-dependent” extension reaction. In order to possess such a capability, the primer must have a 3′-hydroxyl (or other chemical group suitable for polymerase mediated extension) terminus, and be hybridized to a second nucleic acid molecule (i.e. the “template”). A primer is generally composed of a unique sequence of 8 bases or longer complementary to a specific region of the target molecule such that the 3′ end of the primer is immediately proximal to a target nucleotide of interests. Typically, the complementary region of the primer is from about 12 bases to about 20 bases.

Single nucleotide polymorphisms (SNPs) are positions at which two alternative bases occur at appreciable frequency (>1%) in a given population, and are the most common type of genetic variation. The site is usually preceded by and followed by highly conserved sequences of the allele (e.g., sequences that vary in less than 1/100) or 1/1000 members of the populations). A single nucleotide polymorphism usually arises due to substitution of one nucleotide for another at the polymorphic site. A transition is the replacement of one purine by another purine or one pyrimidine by another pyrimidine. A transversion is the replacement of a purine by a pyrimidine or vice versa. Single nucleotide polymorphisms can also arise from a deletion of a nucleotide or an insertion of a nucleotide relative to a reference allele.

Single nucleotide polymorphisms may be functional or non-functional. Functional polymorphisms affect gene regulation or protein sequence whereas non-functional polymorphisms do not. Depending on the site of the polymorphism and importance of the change, functional polymorphisms can also cause, or contribute to diseases.

SNPs can occur at different locations of the gene and may affect its function. For instance, polymorphisms in promoter and enhancer regions can affect gene function by modulating transcription, particularly if they are situated at recognition sites for DNA binding proteins. Polymorphisms in the 5′ untranslated region of genes can affect the efficiency with which proteins are translated. Polymorphisms in the protein-coding region of genes can alter the amino acid sequence and thereby alter gene function. Polymorphisms in the 3′ untranslated region of gene can affect gene function by altering the secondary structure of RNA and efficiency of translation or by affecting motifs in the RNA that bind proteins which regulate RNA degradation. Polymorphisms within introns can affect gene function by affecting RNA splicing.

The term genotyping or genotype refers to the determination of the genetic information an individual carries at one or more positions in the genome. For example, genotyping may comprise the determination of which allele or alleles an individual carries for a single SNP or the determination of which allele or alleles an individual carries for a plurality of SNPs. For example, a particular nucleotide in a genome may be an A in some individuals and a C in other individuals. Those individuals who have an A at the position have the A allele and those who have a C have the C allele. In a diploid organism the individual will have two copies of the sequence containing the polymorphic position so the individual may have an A allele and a C allele or alternatively two copies of the A allele or two copies of the C allele. Each allele may be present at a different frequency in a given population, for example 30% of the chromosomes in a population may carry the A allele and 70% the C allele. The frequency of the A allele would be 30% and the frequency of the C allele would be 70% in that population. Those individuals who have two copies of the C allele are homozygous for the C allele and the genotype is CC, those individuals who have two copies of the A allele are homozygous for the A allele and the genotype is AA, and those individuals who have one copy of each allele are heterozygous and the genotype is AC.

The Example provided herein illustrates the use of genotyping analysis to identify SNPs that can be used to infer parentage of a bovine subject (see Example, infra). Over all allele frequencies (see e.g., Table 2) were determined using extension oligonucleotide primers (SEQ ID NOS: 261-390) to identify particular SNPs in a target nucleic acid sequence. In some embodiments, forward oligonucleotide primers (SEQ ID NO:S:1-130) and reverse oligonucleotide primers (SEQ ID NOS: 131-260) were used to amplify specific target sequences prior to extension.

The oligonucleotide primer sequences listed in Table 1 can be used as “sets” of oligonucleotides. For example, the set of oligonucleotides useful for identifying marker MMIBP0001 can include SEQ ID NO:1, SEQ ID NO:131 and SEQ ID NO:261, or any combination thereof. The MMIBP0001 marker comprises the single nucleotide polymorphism (SNP) corresponding to the first nucleotide, or the complement thereof, in the 3′ position to SEQ ID NOs:261 (extension primer). SEQ ID NO: 1 (forward primer) and SEQ ID NO: 131 (reverse primer) can be used to amplify the sequence contining the marker prior to detection. Thus, each set of oligonucleotide primers provides the means for detecting at least one genetic marker useful for determining the parentage of a subject animal. In another example, the MMIBP0002 marker is identifiable using SEQ ID NO:2, SEQ ID NO: 132 and SEQ ID NO:262. Thus, the “marker set” of oligonucleotide primers for marker MMIBP0002 comprises SEQ ID NO:2, SEQ ID NO: 132 and SEQ ID NO:262. Such a set of oligonuclotides can be designated “marker set MMIBP0002.” In addition, the oligonucleotides useful for amplifying a target nucleic acid sequence would include a “primer pair” such as SEQ ID NO:1 and SEQ ID NO:131 or SEQ ID NO:2 and SEQ ID NO: 132. A “primer pair” includes a forward and reverse oligonucleotide primer while a “marker set” would include a forward, a reverse and an extension oligonucleotide primer.

A SNP was identified as being associated with parentage by determining the probability that a random individual from a selected population (interbreed or intrabreed) was a parent of an animal based on the genotype of one parent and offspring. Table 1 provides primer sequences (See “Forward,” “Reverse,” and “Extension”) that were used to amplify a region that includes the SNP, and amplicon sequences that indicate the nucleotide occurrences for the SNP that were identified in brackets within the sequence.

In another embodiment, the present invention provides a method for sorting one or more bovine subjects, that includes inferring the parentage of a first bovine subject from a nucleic acid sample of the first bovine subject, by identifying a nucleotide occurrence of at least one single nucleotide polymorphism (SNP) corresponding to the first nucleotide, or the complement thereof, in the 3′ position to any one of SEQ ID NOs: 261-390, thereby inferring the identity of the bovine subject. The first bovine subject can be sorted based upon intra- or interbreed (i.e., overall) criteria. The method can then be repeated for additional subjects, thereby sorting bovine subjects. The bovine subjects can be sorted, for example, based on whether they are Angus, Limousin, Brahman, Simmental, Hereford, Gelbvieh or Charolais cattle.

In another embodiment, the present invention provides a method for identifying a bovine single nucleotide polymorphism (SNP) informative of parentage, that includes performing whole genome shotgun sequencing of a bovine genome, and genotyping at least two bovine subjects from within the same breed, or derived from at least two different breeds, thereby identifying the bovine single nucleotide polymorphisms informative of parentage. The Example provided herein, illustrates the use of this method to identify parentage or identity SNPs.

As used herein, the term “at least one”, when used in reference to a gene, SNP, haplotype, or the like, means 1, 2, 3, 4, 5, 6, 7, 8, 9, 10, etc., up to and including all of the haplotype alleles, genes, haplotypes, and/or SNPs of the bovine genome. Reference to “at least a second” gene, SNP, haplotype or the like, means two or more, i.e., 2, 3, 4, 5, 6, 7, 8, 9, 10, etc., bovine genes, SNPs, haplotypes, or the like.

Polymorphisms are allelic variants that occur in a population that can be a single nucleotide difference present at a locus, or can be an insertion or deletion of one, a few or many consecutive nucleotides. As such, a single nucleotide polymorphism (SNP) is characterized by the presence in a population of one or two, three or four nucleotides (i.e., adenosine, cytosine, guanosine or thymidine), typically less than all four nucleotides, at a particular locus in a genome such as the human genome. It will be recognized that, while the methods of the invention are exemplified primarily by the detection of SNPs, the disclosed methods or others known in the art similarly can be used to identify other types of bovine polymorphisms, which typically involve more than one nucleotide.

In another embodiment, the present invention provides an isolated polynucleotide that includes a fragment of contiguous nucleotides of any one of SEQ ID NOS: 261-390, wherein the fragment functions as an extension oligonucleotide in determining the identity of a single nucleotide polymorphism (SNP) corresponding to the first nucleotide, or the complement thereof, in the 3′ position to any one of SEQ ID NOS:301-450. In addition, the extension oligonucleotide primer can be at least 90% identical to any one of SEQ ID NOS: 261-390, or a complement thereof.

The polynucleotide or an oligonucleotide of the invention can further include a detectable label. For example, the detectable label can be associated with the polynucleotide at a position corresponding to the first nucleotide, or the complement thereof, in the 3′ position to any one of SEQ ID NOS: 261-390. As discussed in more detail herein, the labeled polynucleotide can be generated, for example, during a microsequencing reaction, such as SNP-IT® reaction.

Detectable labeling of a polynucleotide or oligonucleotide is well known in the art. Particular non-limiting examples of detectable labels include chemiluminescent labels, fluorescent labels, radiolabels, enzymes, haptens, or even unique oligonucleotide sequences.

In another embodiment, the present invention provides an isolated vector that includes a polynucleotide or oligonucleotide disclosed herein. The term “vector” refers to a plasmid, virus or other vehicle known in the art that has been manipulated by insertion or incorporation of a nucleic acid sequence.

Methods that are well known in the art can be used to construct vectors, including in vitro recombinant DNA techniques, synthetic techniques, and in vivo recombination/genetic techniques (See, for example, the techniques described in Maniatis et al. 1989 Molecular Cloning A Laboratory Manual, Cold Spring Harbor Laboratory, N.Y., incorporated herein in its entirety by reference).

In another aspect, the present invention provides a primer pair comprising any one of SEQ ID NOS: I-130 as a first (forward) primer and any one of SEQ ID NOS: 131-260 as a second (reverse) oligonucleotide primer. A primer pair will prime polynucleotide synthesis of a target nucleic acid region.

In another embodiment, the present invention provides marker sets” of oligonucleotides effective for determining a nucleotide occurrence at a single nucleotide polymorphism (SNP) corresponding to the first nucleotide, or the complement thereof, in the 3′ position to any one of SEQ ID NOS: 261-390. A marker set generally includes a forward primer, a reverse primer and an extension primer. Table 1 provides a list of 130 marker sets.

As used herein, “about” means within ten percent of a value. For example, “about 100” would mean a value between 90 and 110.

The term “haplotypes” as used herein refers to groupings of two or more SNPs that are physically present on the same chromosome which tend to be inherited together except when recombination occurs. The haplotype provides information regarding an allele of the gene, regulatory regions or other genetic sequences affecting a trait. The linkage disequilibrium and, thus, association of a SNP or a haplotype allele(s) and a bovine parentage can be strong enough to be detected using simple genetic approaches, or can require more sophisticated statistical approaches to be identified.

Numerous methods for identifying haplotype alleles in nucleic acid samples are known in the art. In general, nucleic acid occurrences for the individual SNPs are determined and then combined to identify haplotype alleles. There are several algorithms for haplotype reconstruction based on pedigree analysis. These are the Maximum Likelihood methods ((Excofier, L., and Slatkin, M., Mol. Biol. Evol. 12: 921-927 (1995)), the parsimony method created by Clark, A. G., Mol. Biol. Evol. 7: 111-122 (1990) and the phase reconstruction method of Stephens, M., et al., Am. J. Hum. Genet. 68:978-989, 2001, which is incorporated herein by reference). These methods can be applied to the data generated, regarding individual nucleotide occurrences in SNP markers of the subject, in order to determine alleles for each haplotype in a subject's genotype. Alternatively, haplotypes can also be determined directly, for each pair of sites, by allele-specific PCR (Clark, A. G. et al., Am. J. Hum. Genet. 63: 595-612 (1998).

As used herein, the term “infer” or “inferring”, when used in reference to the parentage of a subject, means drawing a conclusion about parentage using a process of analyzing individually or in combination, nucleotide occurrence(s) of one or more SNP(s), which can be part of one or more haplotypes, in a nucleic acid sample of the subject, and comparing the individual or combination of nucleotide occurrence(s) of the SNP(s) to known relationships of nucleotide occurrence(s) of the SNP(s) in other bove animals. As disclosed herein, the nucleotide occurrence(s) can be identified directly by examining nucleic acid molecules, or indirectly by examining a polypeptide encoded by a particular gene where the polymorphism is associated with an amino acid change in the encoded polypeptide.

In diploid organisms such as bovines, somatic cells, which are diploid, include two alleles for each single-locus haplotype. As such, in some cases, the two alleles of a haplotype are referred to herein as a genotype or as a diploid pair, and the analysis of somatic cells, typically identifies the alleles for each copy of the haplotype. Methods of the present invention can include identifying a diploid pair of haplotype alleles. These alleles can be identical (homozygous) or can be different (heterozygous). Haplotypes that extend over multiple loci on the same chromosome include up to 2 to the Nth power alleles where N is the number of loci. It is beneficial to express polymorphisms in terms of multi-locus (i.e. multi SNP) haplotypes because haplotypes offer enhanced statistical power for genetic association studies. Multi-locus haplotypes can be precisely determined from diploid pairs when the diploid pairs include 0 or I heterozygous pairs, and N or N-1 homozygous pairs. When multi-locus haplotypes cannot be precisely determined, they can sometimes be inferred by statistical methods. Methods of the invention can include identifying multi-locus haplotypes, either precisely determined, or inferred.

A sample useful for practicing a method of the invention can be any biological sample of a subject, typically a bovine subject, that contains nucleic acid molecules, including portions of the gene sequences to be examined, or corresponding encoded polypeptides, depending on the particular method. As such, the sample can be a cell, tissue or organ sample, or can be a sample of a biological material such as blood, milk, semen, saliva, hair, tissue, and the like. A nucleic acid sample useful for practicing a method of the invention can be deoxyribonucleic (DNA) acid or ribonucleic acids (RNA). The nucleic acid sample generally is a deoxyribonucleic acid sample, particularly genomic DNA or an amplification product thereof. However, where heteronuclear ribonucleic acid, which includes unspliced mRNA precursor RNA molecules and non-coding regulatory molecules such as RNA, is available, a cDNA or amplification product thereof can be used.

Where each of the SNPs of the haplotype is present in a coding region of a gene(s), the nucleic acid sample can be DNA or RNA, or products derived therefrom, for example, amplification products. Furthermore, while the methods of the invention generally are exemplified with respect to a nucleic acid sample, it will be recognized that particular haplotype alleles can be in coding regions of a gene and can result in polypeptides containing different amino acids at the positions corresponding to the SNPs due to non-degenerate codon changes. As such, in another aspect, the methods of the invention can be practiced using a sample containing polypeptides of the subject.

In one embodiment, DNA samples are collected and stored in a retrievable barcode system, either automated or manual, that ties to a database. Collection practices include systems for collecting tissue, hair, mouth cells or blood samples from individual animals at the same time that ear tags, electronic identification or other devices are attached or implanted into the animal. All identities of animals can be automatically uploaded into a primary database. Tissue collection devices can be integrated into the tool used for placing the ear tag. Body fluid samples can be collected and stored on a membrane bound system.

The sample is then analyzed on the premises or sent to a laboratory where a medium to high-throughput genotyping system is used to analyze the sample.

The subject of the present invention can be any bovine subject, for example a bull, a cow, a calf, a steer, or a heifer or any bovine embryo or tissue.

In another aspect, the present invention provides a system for determining the nucleotide occurrences in a population of bovine single nucleotide polymorphisms (SNPs). The system typically includes a hybridization medium and/or substrate that includes at least two oligonucleotides of the present invention, or oligonucleotides used in the methods of the present invention. The hybridization medium and/or substrate are used to determine the nucleotide occurrence of bovine SNPs that are associated with parentage. Accordingly, the oligonucleotides are used to determine the nucleotide occurrence of bovine SNPs that are associated with a parentage. The determination can be made by selecting oligonucleotides that bind at or near a genomic location of each SNP of the series of bovine SNPs. The system of the present invention typically includes a reagent handling mechanism that can be used to apply a reagent, typically a liquid, to the solid support. The binding of an oligonucleotide of the series of oligonucleotides to a polynucleotide isolated from a genome can be affected by the nucleotide occurrence of the SNP. The system can include a mechanism effective for moving a solid support and a detection mechanism. The detection method detects binding or tagging of the oligonucleotides.

Accordingly, in another embodiment, the present invention provides a method for determining a nucleotide occurrence of a single nucleotide polymorphism (SNP) in a bovine sample, that includes contacting a bovine polynucleotide in the sample with an oligonucleotide (e.g., any one of SEQ ID NOS: 261-390) that binds to a target nucleic acid region and identifies the nucleotide occurrence of a single nucleotide polymorphism (SNP) corresponding first nucleotide 3′ to the oligonucleotide. The nucleotide can be detected by amplification or it can be detected based on the lack of incorporation of a specific nucleotide.

In another aspect, forward and reverse primers can be used to amplify the bovine polynucleotide target nucleic acid using a pair of oligonucleotides that constitute a primer pair, and the nucleotide occurrence is determined using an amplification product generated using the primer pair. For example, the primer pair, is any of the forward and reverse primer pairs listed in Table 1.

Medium to high-throughput systems for analyzing SNPs, known in the art such as the SNPStream® UHT Genotyping System (Beckman/Coulter, Fullerton, Calif.) (Boyce-Jacino and Goelet Patents), the Mass Array™ system (Sequenom, San Diego, Calif.) (Storm, N. et al. (2002) Methods in Molecular Biology. 212: 241-262.), the BeadArray™ SNP genotyping system available from Illumina (San Diego, Calif.)(Oliphant, A., et al. (June 2002) (supplement to Biotechniques), and TaqMan™ (Applied Biosystems, Foster City, Calif.) can be used with the present invention. However, the present invention provides a medium to high-throughput system that is designed to detect nucleotide occurrences of bovine SNPs, or a series of bovine SNPs that can make up a series of haplotypes. Therefore, as indicated above the system includes a solid support or other method to which a series of oligonucleotides can be associated that are used to determine a nucleotide occurrence of a SNP for a series of bovine SNPs that are associated with a trait. The system can further include a detection mechanism for detecting binding of the series of oligonucleotides to the series of SNPs. Such detection mechanisms are known in the art.

The system can be a microfluidic device. Numerous microfluidic devices are known that include solid supports with microchannels (See e.g., U.S. Pat. Nos. 5,304,487, 5,110,745, 5,681,484, and 5,593,838).

The SNP detection systems of the present invention are designed to determine nucleotide occurrences of one SNP or a series of SNPs. The systems can determine nucleotide occurrences of an entire genome-wide high-density SNP map.

Numerous methods are known in the art for determining the nucleotide occurrence for a particular SNP in a sample. Such methods can utilize one or more oligonucleotide probes or primers, including, for example, an amplification primer pair that selectively hybridizes to a target polynucleotide, which corresponds to one or more bovine SNP positions. Oligonucleotide probes useful in practicing a method of the invention can include, for example, an oligonucleotide that is complementary to and spans a portion of the target polynucleotide, including the position of the SNP, wherein the presence of a specific nucleotide at the position (i.e., the SNP) is detected by the presence or absence of selective hybridization of the probe. Such a method can further include contacting the target polynucleotide and hybridized oligonucleotide with an endonuclease, and detecting the presence or absence of a cleavage product of the probe, depending on whether the nucleotide occurrence at the SNP site is complementary to the corresponding nucleotide of the probe. These oligonucleotides and probes are another embodiment of the present invention.

An oligonucleotide ligation assay (Grossman, P. D. et al. (1994) Nucleic Acids Research 22:4527-4534) also can be used to identify a nucleotide occurrence at a polymorphic position, wherein a pair of probes that selectively hybridize upstream and adjacent to and downstream and adjacent to the site of the SNP, and wherein one of the probes includes a terminal nucleotide complementary to a nucleotide occurrence of the SNP. Where the terminal nucleotide of the probe is complementary to the nucleotide occurrence, selective hybridization includes the terminal nucleotide such that, in the presence of a ligase, the upstream and downstream oligonucleotides are ligated. As such, the presence or absence of a ligation product is indicative of the nucleotide occurrence at the SNP site. An example of this type of assay is the SNPlex System (Applied Biosystems, Foster City, Calif.).

An oligonucleotide also can be useful as a primer, for example, for a primer extension reaction, wherein the product (or absence of a product) of the extension reaction is indicative of the nucleotide occurrence. In addition, a primer pair useful for amplifying a portion of the target polynucleotide including the SNP site can be useful, wherein the amplification product is examined to determine the nucleotide occurrence at the SNP site. Particularly useful methods include those that are readily adaptable to a high throughput format, to a multiplex format, or to both. The primer extension or amplification product can be detected directly or indirectly and/or can be sequenced using various methods known in the art. Amplification products which span a SNP locus can be sequenced using traditional sequence methodologies (e.g., the “dideoxy-mediated chain termination method,” also known as the “Sanger Method” (Sanger, F., et al., J. Molec. Biol. 94:441 (1975); Prober et al. Science 238:336-340 (1987)) and the “chemical degradation method,” “also known as the “Maxam-Gilbert method” (Maxam, A. M., et al., Proc. Natl. Acad. Sci. (U.S.A.) 74:560 (1977)), both references herein incorporated by reference) to determine the nucleotide occurrence at the SNP locus.

Methods of the invention can identify nucleotide occurrences at SNPs using genome-wide sequencing or “microsequencing” methods. Whole-genome sequencing of individuals identifies all SNP genotypes in a single analysis. Microsequencing methods determine the identity of only a single nucleotide at a “predetermined” site. Such methods have particular utility in determining the presence and identity of polymorphisms in a target polynucleotide. Such microsequencing methods, as well as other methods for determining the nucleotide occurrence at a SNP locus are discussed in Boyce-Jacino, et al., U.S. Pat. No. 6,294,336, incorporated herein by reference, and summarized herein.

Microsequencing methods include the Genetic Bit™ Analysis method disclosed by Goelet, P. et al. (WO 92/15712, herein incorporated by reference). Additional, primer-guided, nucleotide incorporation procedures for assaying polymorphic sites in DNA have also been described (Kornher, J. S. et al, Nucleic Acids Res. 17:7779-7784 (1989); Sokolov, B. P., Nucleic Acids Res. 18:3671 (1990); Syvanen, A. -C., et al., Genomics 8:684-692 (1990); Kuppuswamy, M. N. et al., Proc. Natl. Acad. Sci. (U.S.A.) 88:1143-1147 (1991); Prezant, T. R. et al, Hum. Mutat. 1:159-164 (1992); Ugozzoli, L. et al., GATA 9:107-112 (1992); Nyren, P. et al., Anal. Biochem. 208:171-175 (1993); and Wallace, WO89/10414). These methods differ from Genetic Bit™ Analysis in that they all rely on the incorporation of labeled deoxynucleotides to discriminate between bases at a polymorphic site. In such a format, since the signal is proportional to the number of deoxynucleotides incorporated, polymorphisms that occur in runs of the same nucleotide can result in signals that are proportional to the length of the run (Syvanen, A. -C., et al. Amer. J. Hum. Genet. (1993) 52:46-59 Other formats for microsequencing include Pyrosequencing (Pyrosequencing AB, Uppsala, Sweden, Alderborn et al (2000)Genome Res. 10:1249-1258).

Alternative microsequencing methods have been provided by Mundy, C. R. (U.S. Pat. No. 4,656,127) and Cohen, D. et al (French Patent 2,650,840; PCT Appln. No. WO91/02087), which discuss a solution-based method for determining the identity of the nucleotide of a polymorphic site. As in the Mundy method of U.S. Pat. No. 4,656,127, a primer is employed that is complementary to allelic sequences immediately 3′- to a polymorphic site.

In response to the difficulties encountered in employing gel electrophoresis to analyze sequences, alternative methods for microsequencing have been developed. Macevicz (U.S. Pat. No. 5,002,867), for example, describes a method for determining nucleic acid sequence via hybridization with multiple mixtures of oligonucleotide probes. In accordance with such method, the sequence of a target polynucleotide is determined by permitting the target to sequentially hybridize with sets of probes having an invariant nucleotide at one position, and variant nucleotides at other positions. The Macevicz method determines the nucleotide sequence of the target by hybridizing the target with a set of probes, and then determining the number of sites that at least one member of the set is capable of hybridizing to the target (i.e., the number of “matches”). This procedure is repeated until each member of a set of probes has been tested.

Boyce-Jacino, et al., U.S. Pat. No. 6,294,336 provides a solid phase sequencing method for determining the sequence of nucleic acid molecules (either DNA or RNA) by utilizing a primer that selectively binds a polynucleotide target at a site wherein the SNP is the most 3′ nucleotide selectively bound to the target.

The occurrence of a SNP can be determined using denaturing HPLC such as described in Nairz K et al (2002) Proc. Natl. Acad. Sci. (U.S.A.) 99:10575-80, and the Transgenomic WAVE® System (Transgenomic, Inc. Omaha, Nebr.).

Oliphant et al. report a method that utilizes BeadArray™ Technology that can be used in the methods of the present invention to determine the nucleotide occurrence of a SNP (supplement to Biotechniques, June 2002). Additionally, nucleotide occurrences for SNPs can be determined using a DNAMassARRAY system (SEQUENOM, San Diego, Calif.). This system combines proprietary SpectroChips™, microfluidics, nanodispensing, biochemistry, and MALDI-TOF MS (matrix-assisted laser desorption ionization time of flight mass spectrometry).

As another example, the nucleotide occurrences of bovine SNPs in a sample can be determined using the SNP-IT™ method (Beckman Coulter, Fullerton, Calif.). In general, SNP-IT™ is a 3-step primer extension reaction. In the first step a target polynucleotide is isolated from a sample by hybridization to a capture primer, which provides a first level of specificity. In a second step the capture primer is extended from a terminating nucleotide triphosphate at the target SNP site, which provides a second level of specificity. In a third step, the extended nucleotide trisphosphate can be detected using a variety of known formats, including: direct fluorescence, indirect fluorescence, an indirect colorimetric assay, mass spectrometry, fluorescence polarization, etc. Reactions can be processed in 384 well format in an automated format using a SNPstream™ instrument (Beckman Coulter, Fullerton, Calif.). Reactions can also be analyzed by binding to Luminex biospheres (Luminex Corporation, Austin, Tex., Cai. H. (2000) Genomics 66(2):135-43.). Other formats for SNP detection include TaqMan™ (Applied Biosystems, Foster City, Calif.), Rolling circle (Hatch et al (1999) Genet. Anal. 15: 35-40, Qi et al (2001) Nucleic Acids Research Vol. 29 e116), fluorescence polarization (Chen, X., et al. (1999) Genome Research 9:492-498), SNaPShot (Applied Biosystems, Foster City, Calif.) (Makridakis, N. M. et al. (2001) Biotechniques 31:1374-80.), oligo-ligation assay (Grossman, P. D., et al. (1994) Nucleic Acids Research 22:4527-4534), locked nucleic acids (LNATM,Link, Technologies LTD, Lanarkshire, Scotland, EP patent 1013661, U.S. Pat. No. 6,268,490), Invader Assay (Aclara Biosciences, Wilkinson, D. (1999) The Scientist 13:16), padlock probes (Nilsson et al. Science (1994), 265: 2085), Sequence-tagged molecular inversion probes (similar to padlock probes) from ParAllele Bioscience (South San Francisco, Calif.; Hardenbol, P. et al. (2003) Nature Biotechnology 21:673-678), Molecular Beacons (Marras, S. A. et al. (1999 Genet Anal. 14:151-156), the READIT™ SNP Genotyping System from Promega (Madison, Wis.) (Rhodes R. B. et al. (2001) Mol Diagn. 6:55-61), Dynamic Allele-Specific Hybridization (DASH) (Prince, J. A. et al. (2001) Genome Research 11: 152-162), the Qbead™ system (quantum dot encoded microspheres conjugated to allele-specific oligonucleotides)(Xu H. et al. (2003) Nucleic Acids Research 31 :e43), Scorpion primers (similar to molecular beacons except unimolecular) (Thelwell, N. et al. (2000) Nucleic Acids Research 28:3752-3761), and Magiprobe (a novel fluorescence quenching-based oligonucleotide probe carrying a fluorophore and an intercalator)(Yamane A. (2002) Nucleic Acids Research 30:e97). In addition, Rao, K. V. N. et al. ((2003) Nucleic Acids Research. 31 :e66), recently reported a microsphere-based genotyping assay that detects SNPs directly from human genomic DNA. The assay involves a structure-specific cleavage reaction, which generates fluorescent signal on the surface of microspheres, followed by flow cytometry of the microspheres. With a slightly different twist on the Sequenom technology (MALDI), Sauer et al. ((2003) Nucleic Acids Research 31 :e63) generate charge-tagged DNA (post PCR and primer extension), using a photocleavable linker.

The nucleotide occurrence of a SNP can be identified by other methodologies as well as those discussed above. For example, the identification can use microarray technology, which can be performed with PCR, for example using Affymetrix technologies and GenFlex Tag arrays (See e.g., Fan et al (2000) Genome Res. 10:853-860), or using a bovine gene chip containing proprietary SNP oligonucleotides (See e.g., Chee et al (1996), Science 274:610-614; and Kennedy et al. (2003) Nature Biotech 21:1233-1237) or without PCR, or sequencing methods such as mass spectrometry, scanning electron microscopy, or methods in which a polynucleotide flows past a sorting device that can detect the sequence of the polynucleotide. The occurrence of a SNP can be identified using electrochemical detection devices such as the eSensor™ DNA detection system (Motorola, Inc., Yu, C. J. (2001) J. Am Chem. Soc. 123:11155-11161). Other formats include melting curve analysis using fluorescently labeled hybridization probes, or intercalating dyes (Lohmann, S. (2000) Biochemica 4, 23-28, Herrmann, M. (2000) Clinical Chemistry 46: 425).

The SNP detection systems of the present invention typically utilize selective hybridization. As used herein, the term “selective hybridization” or “selectively hybridize,” refers to hybridization under moderately stringent or highly stringent conditions such that a nucleotide sequence preferentially associates with a selected nucleotide sequence over unrelated nucleotide sequences to a large enough extent to be useful in identifying a nucleotide occurrence of a SNP. It will be recognized that some amount of non-specific hybridization is unavoidable, but is acceptable provide that hybridization to a target nucleotide sequence is sufficiently selective such that it can be distinguished over the non-specific cross-hybridization, for example, at least about 2-fold more selective, generally at least about 3-fold more selective, usually at least about 5-fold more selective, and particularly at least about 10-fold more selective, as determined, for example, by an amount of labeled oligonucleotide that binds to target nucleic acid molecule as compared to a nucleic acid molecule other than the target molecule, particularly a substantially similar (i.e., homologous) nucleic acid molecule other than the target nucleic acid molecule. Conditions that allow for selective hybridization can be determined empirically, or can be estimated based, for example, on the relative GC:AT content of the hybridizing oligonucleotide and the sequence to which it is to hybridize, the length of the hybridizing oligonucleotide, and the number, if any, of mismatches between the oligonucleotide and sequence to which it is to hybridize (see, for example, Sambrook et al., “Molecular Cloning: A laboratory manual (Cold Spring Harbor Laboratory Press 1989)).

An example of progressively higher stringency conditions is as follows: 2×SSC/0.1% SDS at about room temperature (hybridization conditions); 0.2×SSC/0.1% SDS at about room temperature (low stringency conditions); 0.2×SSC/0.1% SDS at about 42EC (moderate stringency conditions); and 0.1×SSC at about 68EC (high stringency conditions). Washing can be carried out using only one of these conditions, e.g., high stringency conditions, or each of the conditions can be used, e.g., for 10-15 minutes each, in the order listed above, repeating any or all of the steps listed. However, as mentioned above, optimal conditions will vary, depending on the particular hybridization reaction involved, and can be determined empirically.

The term “polynucleotide” is used broadly herein to mean a sequence of deoxyribonucleotides or ribonucleotides that are linked together by a phosphodiester bond. For convenience, the term “oligonucleotide” is used herein to refer to a polynucleotide that is used as a primer or a probe. Generally, an oligonucleotide useful as a probe or primer that selectively hybridizes to a selected nucleotide sequence is at least about 15 nucleotides in length, usually at least about 18 nucleotides, and particularly about 21 nucleotides or more in length.

A polynucleotide can be RNA or can be DNA, which can be a gene or a portion thereof, a cDNA, a synthetic polydeoxyribonucleic acid sequence, or the like, and can be single stranded or double stranded, as well as a DNA/RNA hybrid. In various embodiments, a polynucleotide, including an oligonucleotide (e.g., a probe or a primer) can contain nucleoside or nucleotide analogs, or a backbone bond other than a phosphodiester bond. In general, the nucleotides comprising a polynucleotide are naturally occurring deoxyribonucleotides, such as adenine, cytosine, guanine or thymine linked to 2′-deoxyribose, or ribonucleotides such as adenine, cytosine, guanine or uracil linked to ribose. However, a polynucleotide or oligonucleotide also can contain nucleotide analogs, including non-naturally occurring synthetic nucleotides or modified naturally occurring nucleotides. Such nucleotide analogs are well known in the art and commercially available, as are polynucleotides containing such nucleotide analogs (Lin et al., Nucleic Acids Research (1994) 22:5220-5234 Jellinek et al., Biochemistry (1995) 34:11363-11372; Pagratis et al., Nature Biotechnol. (1997) 15:68-73, each of which is incorporated herein by reference). Primers and probes can also be comprised of peptide nucleic acids (PNA) (Nielsen P E and Egholm M. (1999) Curr. Issues Mol. Biol. 1:89-104).

The covalent bond linking the nucleotides of a polynucleotide generally is a phosphodiester bond. However, the covalent bond also can be any of numerous other bonds, including a thiodiester bond, a phosphorothioate bond, a peptide-like bond or any other bond known to those in the art as useful for linking nucleotides to produce synthetic polynucleotides (see, for example, Tam et al., Nucl. Acids Res. (1994) 22:977-986, Ecker and Crooke, BioTechnology (1995) 13:351360, each of which is incorporated herein by reference). The incorporation of non-naturally occurring nucleotide analogs or bonds linking the nucleotides or analogs can be particularly useful where the polynucleotide is to be exposed to an environment that can contain a nucleolytic activity, including, for example, a tissue culture medium or upon administration to a living subject, since the modified polynucleotides can be less susceptible to degradation.

A polynucleotide or oligonucleotide comprising naturally occurring nucleotides and phosphodiester bonds can be chemically synthesized or can be produced using recombinant DNA methods, using an appropriate polynucleotide as a template. In comparison, a polynucleotide or oligonucleotide comprising nucleotide analogs or covalent bonds other than phosphodiester bonds generally are chemically synthesized, although an enzyme such as T7 polymerase can incorporate certain types of nucleotide analogs into a polynucleotide and, therefore, can be used to produce such a polynucleotide recombinantly from an appropriate template (Jellinek et al., supra, 1995). Thus, the term polynucleotide as used herein includes naturally occurring nucleic acid molecules, which can be isolated from a cell, as well as synthetic molecules, which can be prepared, for example, by methods of chemical synthesis or by enzymatic methods such as by the polymerase chain reaction (PCR).

In various embodiments for identifying nucleotide occurrences of SNPs, it can be useful to detectably label a polynucleotide or oligonucleotide. Detectable labeling of a polynucleotide or oligonucleotide is well known in the art. Particular non-limiting examples of detectable labels include chemiluminescent labels, fluorescent labels, radiolabels, enzymes, haptens, or even unique oligonucleotide sequences.

A method of the identifying a SNP also can be performed using a specific binding pair member. As used herein, the term “specific binding pair member” refers to a molecule that specifically binds or selectively hybridizes to another member of a specific binding pair. Specific binding pair member include, for example, probes, primers, polynucleotides, antibodies, etc. For example, a specific binding pair member includes a primer or a probe that selectively hybridizes to a target polynucleotide that includes a SNP loci or that hybridizes to an amplification product generated using the target polynucleotide as a template.

As used herein, the term “specific interaction,” or “specifically binds” or the like means that two molecules form a complex that is relatively stable under physiologic conditions. The term is used herein in reference to various interactions, including, for example, the interaction of an antibody that binds a polynucleotide that includes a SNP site; or the interaction of an antibody that binds a polypeptide that includes an amino acid that is encoded by a codon that includes a SNP site. According to methods of the invention, an antibody can selectively bind to a polypeptide that includes a particular amino acid encoded by a codon that includes a SNP site. Alternatively, an antibody may preferentially bind a particular modified nucleotide that is incorporated into a SNP site for only certain nucleotide occurrences at the SNP site, for example using a primer extension assay.

A specific interaction can be characterized by a dissociation constant of at least about 1×10⁻⁶ M, generally at least about 1×10⁻⁷ M, usually at least about 1×10⁻⁸ M, and particularly at least about 1×10⁻⁹ M or 1×10⁻¹⁰ M or less. A specific interaction generally is stable under physiological conditions, including, for example, conditions that occur in a living individual such as a human or other vertebrate or invertebrate, as well as conditions that occur in a cell culture such as used for maintaining mammalian cells or cells from another vertebrate organism or an invertebrate organism. Methods for determining whether two molecules interact specifically are well known and include, for example, equilibrium dialysis, surface plasmon resonance, and the like.

The invention also relates to kits, which can be used, for example, to perform a method of the invention. Thus, in one embodiment, the invention provides a kit for identifying nucleotide occurrences or haplotype alleles of bovine SNPs. Such a kit can contain, for example, an oligonucleotide probe, primer, or primer pair, or combinations thereof for identifying the nucleotide occurrence of at least one bovine single nucleotide polymorphism (SNP) associated with parentage, such as a SNP corresponding to the first nucleotide, or the complement thereof, in the 3′ position to any one of SEQ ID NOs:301-450, following hybridization and primer extension. Such oligonucleotides being useful, for example, to identify a SNP or haplotype allele as disclosed herein; or can contain one or more polynucleotides corresponding to a portion of a bovine gene containing one or more nucleotide occurrences associated with a bovine trait, such polynucleotide being useful, for example, as a standard (control) that can be examined in parallel with a test sample. In addition, a kit of the invention can contain, for example, reagents for performing a method of the invention, including, for example, one or more detectable labels, which can be used to label a probe or primer or can be incorporated into a product generated using the probe or primer (e.g., an amplification product); one or more polymerases, which can be useful for a method that includes a primer extension or amplification procedure, or other enzyme or enzymes (e.g., a ligase or an endonuclease), which can be useful for performing an oligonucleotide ligation assay or a mismatch cleavage assay; and/or one or more buffers or other reagents that are necessary to or can facilitate performing a method of the invention. The primers or probes can be included in a kit in a labeled form, for example with a label such as biotin or an antibody. In one embodiment, a kit of the invention provides a plurality of oligonucleotides of the invention, including one or more oligonucleotide probes or one or more primers, including forward and/or reverse primers, or a combination of such probes and primers or primer pairs. Such a kit also can contain probes and/or primers that conveniently allow a method of the invention to be performed in a multiplex format.

The kit can also include instructions for using the probes or primers to determine a nucleotide occurrence of at least one bovine SNPs.

Many software programs for molecular population genetics studies have been developed, their advantage lies in their pre-programmed complex mathematical techniques and ability to handle large volumes of data. Popular programs used by those in the field include, but are not limited to: TFPGA, Arlequin, GDA, GENEPOP, GeneStrut, POPGENE (Labate, J. A., Crop Sci. 40: 1521-1528. (2000)) and Structure. The present disclosure incorporates the use of all of the software disclosed above used to classify bovines into populations based on DNA polymorphisms as well as other software known in the art.

Structure has been used to determine population structure and infer assignment of individual animals to populations for livestock species including poultry (Rosenberg, N. A., et al., Genetics. 159: 699-713 (2001)) and bovines from South Asia (Kumar, P., Heredity 91: 43-50 (2003)).

The following example is intended to illustrate but not limit the invention.

### EXAMPLE

### Identification of SNPs that can be used to Infer Parentage

SNP markers were identified from proprietary whole-genome shotgun sequencing of the bovine genome licensed to MMI Genomics. Over 700,000 putative SNP markers were identified from assembly of fragments and over 200,000 of the putative SNP markers were syntenically mapped to Celera Genomics' working draft of the human genome. The 778 SNP markers were selected for study based on their dispersion pattern throughout the bovine genome as determined by human location, and all markers contained a guanine/adenine purine transition for ease of assay development. Individual markers were tested to determine parentage specificity within the cattle population using 204 animals representing diverse breeds (Angus, Charolais, Limousin, Hereford, Brahman, Simmental and Gelbvieh). 130 G/A SNP markers that have minor allele frequencies between 0.2 and 0.5 for the major cattle breeds were identified. These markers can be multiplexed because of the common extension to create a powerful panel that can be used for identity or parentage verification in a number of breeds.

The SNP detection platform used was the SNP-IT™ system(Beckman Coulter, Fullerton, Calif.). In general, SNP-T™ is a 3-step primer extension reaction. In the first step a target polynucleotide is isolated from a sample by hybridization to a capture primer, which provides a first level of specificity. In a second step the capture primer is extended from a terminating nucleotide triphosphate at the target SNP site, which provides a second level of specificity. In a third step, the extended nucleotide trisphosphate can be detected using a variety of known formats, including, for example: direct fluorescence, indirect fluorescence, an indirect colorimetric assay, mass spectrometry, and fluorescence polarization. Reactions were processed in an automated 384 well format using a SNPstream™ instrument (Beckman Coulter, Fullerton, Calif.).

Specifically, markers were assayed on Beckman Coulter GenomeLab™ SNPstream® Genotyping System. Markers were amplified in a 5 ul reaction volume of a 12-marker multiplex in a 384-well format. The PCR is performed as follows: 95° C. for 10 min., followed by 34 cycles of 94° C. for 30 s, 55° C. for 30 s, and 72° C. for 1 min. The DNA products are cleaned using 3 ul of diluted SNP-T™ Clean-Up (USB), incubated at 37° C. for 30 m with a final inactivation step of 96° C. for 10 min. The extension reaction is performed as described by the manufacturer, with 0.2 ul of the G/A extension mix 3.762 ul extension mix diluent, 0.021 ul DNA polymerase, 3 ul of extension primer working stock, and 0.018 ul water added to the 8 ul volume in the well after clean-up. This 15 ul extension reaction is then thermal cycled as follows: 96° C. for 3 min, followed by 45 cycles of 94° C. for 20 s and 40° C. for 11 s. Following extension, 8 ul of hybridization cocktail is added and mixed. Ten microliters of this mixture is then transferred to the 384-well SNPStream® Tag Array plate. The plate is then incubated at 42° C. for 2 hr. Each of the 384 wells in a Tag Array plate contains 16 unique oligonucleotides of a known sequence, or tag. After hybridization, the Tag Array plate is then washed, dried (1 hr), and read on the SNPstream® SNPScope Array Imager. The raw image data is then analyzed and genotype calls generated using the software provided, then reviewed by scientists before data is uploaded into the database.

Each marker was evaluated in 8 breeds of cattle: Holstein, Brahman, Angus, Hereford, Limousin, Simmental, Charolais and Gelbvieh with 20 to 27 animals per breed for a total of 204 individuals. In addition, markers were tested for Mendelian inheritance using trios of 20 animals. Allele frequencies were determined within breed and overall. Exclusion probability at any locus I, (Ql), is the probability of excluding a random individual from the population as a potential parent of an animal based on the genotype of one parent and offspring. Following Weir (Weir, Genetic Data Analysis II. Sinauer, Sunderland, Mass.)
Qₗ=pₗ−2pₗ²2+2pₗ³p−ₗ⁴

where pl is the frequency of the guanine allele at locus l. The overall probability of exclusion is one minus the probability that none of the loci allows exclusion and is calculated as

$$Q = 1 - {\prod }_{l} ( 1 - {Q}_{l} )$$

Match probability ratio (MPR) was calculated, using the ceiling principle, as the square of the most frequent allele frequency to provide the most conservative estimate of match rate within a breed. Overall match probability ratio was estimated as the product of MPR at each SNP marker.

Table 1 lists the primer sequences for each of the SNP markers including PCR primers and extension primers. All SNPs are G/A purine transitions. Table 2 lists the allele frequencies within each of the breeds studied, the number of observations recorded for each breed and the standard error of the allele frequency estimate.

Thus, the oligonucleotide primer sequences listed in Table 1 can be used as “sets” of oligonucleotides. For example, the set of oligonucleotides useful for identifying marker MMIBP0001 can include SEQ ID NO:1, SEQ ID NO:131 and SEQ ID NO:261, or any combination thereof. The MMIBP0001 marker comprises the single nucleotide polymorphism (SNP) corresponding to the first nucleotide, or the complement thereof, in the 3′ position to SEQ ID NOs:261 (extension primer). SEQ ID NO: 1 (forward primer) and SEQ ID NO: 131 (reverse primer) can be used to amplify the sequence contining the marker prior to detection. Thus, each set of oligonucleotide primers provides the means for detecting at least one genetic marker useful for determining the parentage of a subject animal. In another example, the MMIBP0002 marker is identifiable using SEQ ID NO:2, SEQ ID NO: 132 and SEQ ID NO:262. Thus, the “marker set” of oligonucleotide primers for marker MMIBP0002 comprises SEQ ID NO:2, SEQ ID NO: 132 and SEQ ID NO:262. Such a set of oligonuclotides can be designated “marker set MMIBP0002.” In addition, the oligonucleotides useful for amplifying a target nucleic acid sequence would include a “primer pair” such as SEQ ID NO: 1 and SEQ ID NO: 131 or SEQ ID NO:2 and SEQ ID NO: 132. A “primer pair” includes a forward and reverse oligonucleotide primer while a “marker set” would include a forward, a reverse and an extension oligonucleotide primer.

TABLE 1

The oligonucleotide marker sets for each of the SNP markers including PCR primers and extension
primers is provided. All SNPs are G/A purine transitions.

| | | |
|---|---|---|
| Forward Primer | Reverse Primer | Extension Primer |

| | | | |
|---|---|---|---|
| Marker | (SEQ ID NOS:1-130) | (SEQ ID NOS:131-260) | (SEQ ID NOS:261-390) |

| | | | |
|---|---|---|---|
| MMIBP0001 | TTTACCTACCTCATAAAAATGCTCT | TAGCTAGTGTTGAATTATCATTATCGA | ATGAGTTCATATGAGTAAAGATGCT |
| MMIBP0002 | TCCCGCATCCCCACTTCT | ATCTTGAGAAGCACTGAGGC | TTAACAGCATCCTCCCCTCGGCAAA |
| MMIBP0003 | TGCCAGTCTGAAGAAACCA | TGTCATTTCTGAGTGTACTGGAGA | CATCTTCATTCACAGGGAGAAAACA |
| MMIBP0006 | TGTTTGTATCTTCCAAATTTCATA | CTCCGTGGTCAGGCTCTC | GCAAGGGCATAGTCTTCTTTATGGG |
| MMIBP0007 | TTATGTAATCCCAGGGATGTTG | AATCGCATTTCAAAAATCACC | CAGGAACAACCTCAGTACATACAAC |
| MMIBP0008 | TTTTAGTCTGAGTGTAAATAACTTGGG | AAAGAAAATCAGAAGATGGGAAA | TTGGTCTCTGCTGAACAGCCCGACA |
| MMIBP0009 | AAATAACTCCGTGAATGTGTGG | TTTTCCCAGAACCATTTATTGA | TCAATCTATGATGAAGGAGGCAAGC |
| MMIBP0010 | TTAAAGTGTGGAGCCTGGAG | TTAAAAATCACATGTATGTTTTCCC | ATCTCAGGGGACTTGGGGGTTTCGC |
| MMIBP0014 | AAATTGACTRACTGTTTTTTGTCAC | ATTTAAGGTAGATGCCAGGAATG | AGAAATGCTGTTTTTCTCCTGACAC |
| MMIBP0015 | AAGTGCAAGGTCTTAACCACTG | TCTGAGCTGAGCAAACAGC | AGTCCTAAGCTTGCCTACCTTC |
| MMIBP0016 | ATTATTATCTTGTTTTACTTTGGTAAGAGAC | TGGGCAGTTGTTTTATTTTTTAA | AACAAAGGGAACTGTRAGTTGATCT |
| MMIBP0017 | TCAGGTGATTGCCGTTGT | AACAGTATTCTGGGGACTTGC | CTTTTCAACCCAAGTGGAAACCCAG |
| MMIBP0018 | ATTTCCTACTTTTGCATTACCCA | AAGGAACCAAATGTCTTGGC | GCCCCCTTGACAGTGAGACTTCCTT |
| MMIBP0019 | AAATGATAGTTGTGGCAGTATATA | CATGATTCTTTTATGACTAGATATTGAATG | TGTATTTTAAAATAAATTACAAGCA |
| MMIBP0020 | AATCTGTTTCTGAGCTTGTTCTTG | TCTTGTAAACAGCTGGCTGC | CAAATGGCCCGTAAAGCAMGTGTGC |
| MMIBP0021 | CTTAAAACATGTATTTGTCTTTCTACTT | TAACACTGATGGATCTGGTATGAC | AGATAGTGTGTCTCTTGAGCACTGA |
| MMIBP0026 | ACACACTGCAATTAATAGAGGATTC | CRGTATATTGTMGCAGTTACAGCT | CCCTGCTCTCAAAAGCCACGTAGAG |
| MMIBP0027 | AACAATAAAATGTCATGTAYGTCAA | AATTAAAAACAAGCCAATCTGG | GGGCTGTGAAGATAGGACCAAGTAT |
| MMIBP0029 | TTCTCTCTGGACTCTGTGCAG | GTTGTTGCAWAGTTCTTCTAGGG | TCCCTGTGTGCTAAATTCACATAGC |
| MMIBP0031 | AAAGAAAGGGTGAGGGTGAA | AAAAAAGAAATCTTCCTTCCCTT | ACGAGGGGACGAGAATCAGGCTGAG |
| MMIBP0032 | ATAAGATGCTGGCTGAGACCT | ACCTTCTTAACTTCTGCCTAAACTATT | AAGAGAGGGAAATGTATCATTGGCA |
| MMIBP0034 | AGACCGTCAGGAGCTGAG | ACGTATTTGTAGCTGTTTGTACG | CTCGCCAGATATTAGATCAACAACC |
| MMIBP0036 | ACCTGTTTGCCATCTTCTTTC | AGCCCAACAAGAAAGAGGA | TTTCGTTGGCCTTGCGCTCTCCATC |
| MMIBP0038 | TTTTGGCATACATCAACTTGAA | TATTAGAATCTCAGGGAGAGGGA | CYGCAGGTATACATGGGTCCATTCC |
| MMIBP0040 | CCCAAACTAAAAATGATTTAAGAAAC | ACACAATCCCATGAACAGTAAAA | CAATATTCRTACCAGATAAATTCCA |
| MMIBP0041 | TCTGCAATCTGGCATTGAG | ACTGACTGTAAAATCCTGAGCAG | ACGGTGCCCTGGACTGCAAGGTGCC |
| MMIBP0046 | ATATCCATCCCTTTCTCATCTGT | ACAACCCTAGGTCAGAGATGG | CAGAGCCAAGCCTCCATGAACCCAC |
| MMIBP0047 | ATCTTTCAGTCATGCCAGATC | TTTATGGGAAATTGGTTATGACTT | GCAACGAGAGAAAGACTCATATAGT |
| MMIBP0048 | GGCAGTCACTGACTCTGTAATAGG | ACACAGCACCAGCATGATG | CTCAGATCCATTTCAGTAGCTCATC |
| MMIBP0049 | CTGCCCTCTTCTCCAACC | CACCTGGAGATATTTGATTCATG | TCCCCAAGCCCCCAACCCTCACTCC |
| MMIBP0051 | ACGAAAAGTGCTTTGTGAAAA | GACTGTTTCAGTACTGTTTTCTTGTTT | TTCTTGTTTGCAGTATTGCTTGGTC |
| MMIBP0052 | ACTTGTTGAAAAACTCTAAAGGTAAATT | AAGTACTTTGAAGGATGTAATGCTTAT | ATGCTTATCTCTGGGGAAAGTATGT |
| MMIBP0053 | TGAATGAGCAAAGGTCAGG | CTCTACTTCTATTTCAATCTCCATCAT | CCATCATKAACATCATTGATGCTCA |
| MMIBP0054 | TCCACCTGCTTCCTCTGG | AATTTGGAACCAATTTGGTAATAT | GTWGTATACATATAAACTCATRGAT |
| MMIBP0055 | AAACTGAAGGTTCTTTTTGGTATAGG | TTTGATCTCACCCCCTTCC | AACAAAAACCATGTCAGTCAAACAT |
| MMIBP0056 | GATATAGAGGACTTTTACGAGTTTCATT | ACAGAAAGCCAGATTGTATAACTTTC | TAACTTTCCATTGATACATAGATGC |
| MMIBP0057 | GTAATACTTCATGTAGATTTTTAAACTTTGAG | TTTCCCATATCTGTTGCTCC | AACTTTTACTTTCGAGTCTTGAGGG |
| MMIBP0058 | GAGGTATATGATGAAAACAGCTTAGAG | ATTCCATACTGCATAACACATTTCT | TGTATGTTTTCCCATTGCATTAAAT |
| MMIBP0059 | AAAAGTAACTTACTGAACCAATATTGACA | TGAAATCATATCAGTGGACTTTTTAA | TTTTAATAATGTTATGTTAAAATCC |
| MMIBP0060 | ATTGGGGCATGAACACTG | ATTCACAAATGCTCTGTGCC | CCGAAGCAGATTCAGGGCCCTCCAA |
| MMIBP0061 | ACTCCGCATCCCTGGACC | AACATCCCATCAGTGGTCC | CAACGGGCATMCACAGAGACCCCAT |
| MMIBP0062 | CTGGGACTGAAAGGGGAT | ATGTCCAGGCCTCTCCCA | TGCGGATCCAAATGCTCCCAACAGC |
| MMIBP0063 | AGCAGAGTCCCAGGGCAG | GGCTGAGGACTGTGGAGC | AGATACAGAAGATGCAGGAGGAAGA |
| MMIBP0064 | TTGAACAAGAGGATGATATTCTGC | AAAATACTGTTAAAAAGGGTCTTCTTG | ATACACGATGCTTCCCTATGGTAAA |
| MMIBP0065 | AAGGTGGAACAAAAGCAGTATT | AATGCTGTGTCTGGGAAGAG | CTGACAGGCAAGTCCTCTGATCCTC |
| MMIBP0066 | CTTTTCCTTTTGGTCCTCTG | ACYTGATACAGCGTGTGGAC | ACGTAGGCACTRTCAGGGGAGGTAC |
| MMIBP0067 | AGGAGATATATGTTTGAAATTTAGGTCA | AGCCTGTGGGTCTGAGTCA | CTCGAGCCCAGTGACCCCCCTCATC |
| MMIBP0068 | AAATTTAAACAGAATTCCTACTTAGCA | TTCTTGCATATATTTTATTTCTTTCCC | TTTCCCAGTACATACTATTGTGCTT |
| MMIBP0069 | TGAACCAGATTCCACCTCA | CCAAGAGGCCTAGAATCTCC | CAGACAAGTTGTCCCAGCCCTGCCG |
| MMIBP0070 | AGAAACTGGAACTGCTCGA | AAAACATCTGAAAATTGCACAG | TGGCAATGTTCCTGATTGTTCC |
| MMIBP0071 | ATTCCAGAAGTCTGTTTTAAAATGTC | TGTAACCCTCTGTTGTGTAGTATACG | GGAGTTTTCTTTTTATTCCTGTATG |
| MMIBP0072 | TTATTTACTGTTTGCTTCTGTTATTTC | TTATATTCTGGGGACATMTTGCT | ATCTGGTCCACAATCCAGACAGTTC |
| MMIBP0073 | AAAYAAGATGACCATTAGGTTGATG | TCTCTGTCATTGGTAAGTTCTGG | GTTGGCATGACAAGGATCTGGGTCA |
| MMIBP0074 | ACGAGTGAATGAAGGGAAC | ATGGTAGGAACTACAGAATTGTATTTAATAT | CACTGTTCACCRAGCAAACGGAATG |
| MMIBP0075 | AATATGAAAGTTCTGTAAGTATAAAACAGTGT | TAGGACCTCCGTAATCTCACC | TGAATGGGAAGTGGGTGTGATGGAA |
| MMIBP0076 | GCAGCCCAGTATAATAATAATAGCTC | ATAGGGTTGTAGATTAGAATGAAATGA | ACCCTCTTTRTGTGCCAGATTAAGT |
| MMIBP0077 | TCAAATGCCTACCCTGGTG | TGGAAAGACTATTAGGTCATAGGTTATT | GCCAGAAGAAAAATGTATGCAATAT |
| MMIBP0078 | AATGTTTGGCTACTAGAGTGAGTGA | AGTGTAGCTAGTAGGTGTTTGTCTCTC | ATCTCAAATTGGAAGAAGGTTTTTA |
| MMIBP0079 | ATATATTGCCAATARTGATCACTTTCA | AATTCGCATTGAGGAAAAATG | GATCTGGAATGTGGTAGTGAYTAGT |
| MMIBP0080 | ATTTTTGAATTAGAGCCTTTGACA | AAAAAATTACAGGACATGCCAA | AAYGTCCAYRTTGTTCGAGAATCTC |
| MMIBP0081 | TTCATCCCCTAAAAAGGAGC | AGCAGGGGCTTTAGAGCA | CTGGTCCAGGAAGATCCCACATGCC |
| MMIBP0082 | ACTGCAAATGGCAAGGAA | GTTTGCGCTATTGCTTCTG | TAAATTGAGAGGAAATGATRAAGTG |
| MMIBP0083 | AAAGACACTTCCACCTAGTTCTCC | TACATAGAGTAATACTTGGCTACATGAGTT | GATTAACCCTCAAAAACTGAAAGCA |
| MMIBP0084 | TTTTCTGAAATAATTCCCACCA | TAATACCTACATTTACAAGAACCTTCATT | TTCTCACAACATGCTTGTCTTTACT |
| MMIBP0085 | TATTAAGAAACTACTCGCAGATGTGA | ATAAGAGTTGGTCAAAAGTGGGT | AGTGTGTGTGTGTGAAATCAGCAGA |
| MMIBP0086 | TTCTTATTTTAGACACTATCTCAAGCAT | AATGAGGATTCCTTTCATTATTAATTC | CAAGCATTATTTTAAACAGGCAAAC |
| MMIBP0087 | TACGATGTGTTCACATAGCCAT | AAATGCCATAGTCACTCCAAAG | TTTTTCAACTTTGCAAAAGTAAAAC |
| MMIBP0088 | CCCAAAGGGTAAAATGGC | TTAATAGAACAAAATGAGGAAAACTCTA | CAATATTGGACAATTTGTTAGTAGC |
| MMIBP0089 | ATTCAATGGCACTAAGGCAG | TTCCAAAGTAAGACATGAAAACC | ATAAGTGAACAGGGGGTTTGGTGTG |
| MMIBP0090 | AAAACAGAACAACTACTTGCCTG | AACATGCTTTGAGAATGTTGTG | ACGGATGAGATTCATTTGAACTGGC |
| MMIBP0091 | CTGCTTGCTGTTCAGATAACG | AAATCTGACAAACATTTTCGTGA | AGTGATTTTTCTGGAGCCAGACTGC |
| MMIBP0092 | AACGGCTTGGCAAAGGTA | GGGTCACTCCCTTCTTCTCA | CCAGCRTGAGAAATACTGACRGTGA |
| MMIBP0093 | TATTATAGCTTCTTTCAGAGCTGGG | TATTTGTCACTAAGAATGAGTCAGTATAGA | TTATCTCCCAAAAGATAGAGCTTCA |
| MMIBP0094 | TACATATCTTATATATTCAGGATCCCT | AAAAAACAATAGCTCTCAGAGGAC | TTGCCTTGCTTCGTTTGTTATGAGT |
| MMIBP0095 | ATCTTCCTAATGCCACTTTTATTTAT | TAAGGAAAATCCTTAATCTTATCAGC | AATAGATTATTTTCTGGAGAATACC |
| MMIBP0096 | AATGGTTGACTGCCATGATG | TTAAATGATGCCATGCTTTACTG | GATCAGAGGAGACAATGTCTGTTGG |
| MMIBP0097 | ACCAAAAAAAYTCACAATAAGCC | TCCTAGAGAAGTTGAGCCATCA | GTTACCAGTTTAGGGAACCTACCAC |
| MMIBP0098 | ATGAATGTTTGACTTTTGAATTGT | ATTTTGGCTGCAGAATGG | AGTGAATTTGTACAAGGCTTCC |
| MMIBP0099 | ACTTGGTGACTGAACAACAAAAT | TTTACTTGAACAGGATTTGGTTTAG | TAAGAACCCAGACATTTTTACAAAC |
| MMIBP0100 | TTTCTCATCCCTCCCCCT | AAATGATTAATGATGGATTTTCCA | AATATTGATCTTGTGTAGTATATGC |
| MMIBP0101 | TGTGCTCCGTGTTCCAGA | AAAGAAACTGCTTTCTATGGTAGACA | TGTCTGGGGAACTGAGGTCAGCCGC |
| MMIBP0102 | CTGAACTGAGAAGGAGGGA | ATACTGTTTCTGAGGCAGCTG | AGCAGTGAGTTTACTTTATGGAATA |
| MMIBP0103 | ATATGTTGATGTCATAGTACACCCC | TCATATGCTAATGACCTCATTTTAAA | ACTTCAGAATGTGGCCATATTTGGA |
| MMIBP0104 | TTAGGTTACCAGGTGTAGCCC | CATTTTTTAGTATAAGTATGTTTTGAAGACTG | AGAAAAGGAAAACACATACACACAC |
| MMIBP0105 | TGGCACAGCATCTTGTCTC | ACGTCTTTTTGGATTGATAGGA | ATTTAAACATTTCCTTTTAGATTGA |
| MMIBP0106 | CATTTAAGTTGTCCACCTATGAAGT | AGTCTCCTCACCGTTACTTGAG | CAGCCTAGGTTGAGACATTCAGCAG |
| MMIBP0107 | AAATTATAAAAAAGCATTCTAGTCAGAGTC | TCACATGTAAAACCACAAAAACA | TTTAGTGTATAACAGTTAAAAATGA |
| MMIBP0108 | TTAATTAATTACAAAATGCAGCTGTG | TTGGTACATATTCACATACTTTTTTTCT | CATCCTTCAGAAAAATGCCAGTGAC |
| MMIBP0109 | TGAATTTCTACCTCAATTTCTAGCC | AAATATCCTGAATGCTTAAAATGAAG | ATCTGCAATTTAAAATGGTGGCATG |
| MMIBP0110 | ATGGTCACCGGACACAGC | TTGATCACTGGAATGAAACTCA | CTTCCAACTGAGCAAATAAAGTTTC |
| MMIBP0111 | AATACATCAACCAGCTTAGGTGTT | AGTCAGCAAGAGCCCAAG | CGTTTCTCTGGAATTTCCTATTCTT |
| MMIBP0112 | CTGAAATTATTCACATATTCACTATAAGC | TTGTTGTTCGTGCAGGTTT | AATCGAGAAATGAAAATAATGGAGG |
| MMIBP0113 | AACATGATCCCCCTCTTACTG | TGTGAATCCCAGGGGAGT | ATTATGATCTATCAGAATGATTTAC |
| MMIBP0114 | TGTCCAAGTCTCTATGTTTCTG | TTACGTATCAAGCCAAAAGAGG | CTTCACAAATAAAATTCACTCAATC |
| MMIBP0115 | ACACATAACAGATTTCCTAATTTG | CACAGATGACAAAGTATTAAAATTATAGC | ATTGTGATTTTTCAAATGTTTGTCA |
| MMIBP0116 | TTTTTAGAAATCAATAAGACAGGTGA | TTTCCCTGGCTACTGGCA | GTAGATCAAAGGAAGTGCAGATGCC |
| MMIBP0117 | ACAAAATAATGCAAATATAATCCTCC | TAGACATAAATTCTAGCAGCAACATT | AGAGAAACRAAGTGCTGTTTTCAAT |
| MMIBP0118 | TTATTAACTGTCTATTACATGTTAGGGTAA | AAAATGTCTACTTTTCAGGTATATTAGGA | CACTGAAATGAAACCTCTAAATACA |
| MMIBP0119 | TATCAATGTCCTTTTTTACAACTTTC | ATAAGGCTCACATAATAGTGGATG | AGATGAAAGTGAATGATAAGCATTT |
| MMIBP0120 | AATTGAAGAGGAAGAAAATTGG | ACTTTTCACCACTCAGAAGGAT | AGAGGACATGGAGGCAGAATGCAGA |
| MMIBP0121 | AAATCATGAGTTGGGGTCTTC | AGGCTGTCATGCTTCTTCAT | GTCTTGGCTCCCTATGACCGTGTCA |
| MMIBP0122 | GAAGTTAACTCCAAAGCAGAT | AGAATCATTATTAAGCATTAAGGTAAGTATG | TTCTTGAAGATTCTGTTACCATTAT |
| MMIBP0123 | TCTACACTATCAAAATTATCATATTTTACCTC | GCCAGGTTAGTCTAATGTTTCAA | TTTATTTGTAAGCATGGTGAATTAC |
| MMIBP0124 | TTAAAAGGAAAGTCTGCTGCTG | ATGAATCCTCTGCCACACA | TTCGTGGGTTGTTCTTCCTGTTTGC |
| MMIBP0125 | TTGCTAAGTCTTTGGGAATCTC | GGCAGATGGTTCTGAATTTAAA | TCAGTACATAAACAGAGTCATTGCC |
| MMIBP0126 | TCAGAAAGGGCATACATCAA | AAAGACAAGCAAAAGGGAGAA | GTGTTAACAACATTTGCATCTCTGA |
| MMIBP0127 | AGGAAGATGAGTCACCGGT | TAGACTCTGCCATGCGTCA | AGCAAGTCAGTCTGTGGAGGCGGCA |
| MMIBP0128 | AGAGAATCAGGCACAAGGC | ACACCCCATCTCCTCTACCT | TACAGCAACTATTATTCAATCTTTT |
| MMIBP0129 | ACTGACACCTCCATCCATC | GAATTTTCTTCCACTTAGAAAACCT | GAGAGATAGGTTCATAAGCTTGTTG |
| MMIBP0130 | CTCCATAATGAACAAAACCCT | TGACTTTCTTTTTTTCCTTAGCAC | GCCCTCCTGCAAGTTAGGTTCTTTA |
| MMIBP0131 | AAAAGGAAGTCTTATTCAGGTGATAG | ATAAACTGTGCGTCCTGAGAG | ACAAGGTGTGCCCTGAAATAAGAAC |
| MMIBP0132 | TACCACAGAGGAAACTTTGG | CSARGTCACATATAGGAATGAAG | TTAAAGTGCTGAAAACGAAAGCTGG |
| MMIBP0133 | TATATTGAATTTAAATGGCTCACCA | TAAACACTGTGATCTGATATTATTAAAACC | TATTTGGAAAATTCTGATACAAAGA |
| MMIBP0134 | TAGAGAAAAGTGGCGCAGC | TGAATCTGTGCTTGTAGTCTTTTTT | TGAAGACCCAGCACTGCCAGAAATA |
| MMIBP0135 | TAAGAAAGGTTAATTAGGAAGAGAAGC | AATTTTCCAGCCTTCAAAACA | GACTCTTAGTCCAGACTTTTCTGAC |
| MMIBP0136 | TCCTACTAGGTGACTAGTATATCTGTACATG | ATAAGTAGAAGCACTTCATTACTTAGCC | AGCTGGTTTTATTTTCCTTCTTTCC |
| MMIBP0137 | AATTTCCTTTTTATCCAATGCC | ACAAACAGGTAGAACACAAGATTTT | TGACATATATCCATCAATATAATAC |
| MMIBP0138 | ACACATCACACAGCCTCCC | TAGTTGATGAGGATGGAGTCTGA | GTAATCTCAGGCAGGGCGGGTAATG |
| MMIBP0139 | AAAATTTGGTGCTTTGATCACT | TATAAAGTGAATGAAAAAAGGGAGATA | CATTCCAATGGCATCAAATGCCTCC |
| MMIBP0140 | TAGATGTGGTAAACAACGAAGAGTAA | ATAGACTGTAGATGGCCTAAGGAC | TCTTACCCACTCTTCCATCAGCACC |
| MMIBP0141 | TGCCACATGCGAGGACTA | GGGGCAAAGCTAAATGGC | CATCTGCACAGTAAGAACAGCGAGC |
| MMIBP0142 | TTTGAAGAAAAACATTACTGGG | ACAAAAGCCGTGAACTTGAG | GACATGAGAAAGATAAAGACCTCAA |
| MMIBP0143 | ATTTCAAACAGCACAGAAGTTATAGG | GCTTAGAGAGATAGTTGAGGGCA | CCAGTCCATCTCCACCAGGAGCCCA |
| MMIBP0144 | TAGCTGAGTCCAGTCTAAACTCCT | AATCCACATGCCTACCTTAGG | CCCAGGCCACAGTGTCCATGTACCC |
| MMIBP0145 | TGTGATCTATTTGGTTTGATGAG | TCCTGTACCTGCCTTGATCT | CCTCTTCCCATCCAATCTACATAAC |
| MMIBP0146 | AGAGGACAGGGGGACCTG | ATCTCACCTGCTTTCTTAGATGC | CGGATTTTTCAAGACTCCCCTACGCC |
| MMIBP0147 | AACTGCAGTGCTTGAGGG | GAYCACCCCGCCTTGTCTA | GGAGCTGGAGGAGGTGCAAGACGAC |
| MMIBP0148 | GGATGGCAGAGTCCAGCT | GCCTTATTGTTTTTTATTTCATGATC | GGGCGAGAGTGCAGGAGCTCAGGGC |
| MMIBP0149 | AAAAAACAAGAAGTGCAAGAAGTC | ACTTCCTCTCTGTTAGGGATAACAT | CTTTCCTCCCCACAAAAGAACCTAA |
| MMIBP0150 | TAAAGTTTACATTTTTTCCCACCA | TAAGTTTGATGGATTTTTCCTACTATG | CCTAATTTAGCTTGAAAATGAGTTC |

TABLE 2
The allele frequencies within each of the breeds studied, the number of observations
recorded for each breed and the standard error of the allele frequency estimate is provided.

| | Angus | No. of | | Brahaman | No. of | | Charolais | No. of | |
|---|---|---|---|---|---|---|---|---|---|
| Alias | G Freq | Gametes | SE | G Freq | Gametes | SE | G Freq | Gametes | SE |
| MMIBP0001 | 0.315 | 54 | 0.06 | 0.389 | 54 | 0.07 | 0.731 | 52 | 0.06 |
| MMIBP0002 | 0.111 | 54 | 0.04 | 0.926 | 54 | 0.04 | 0.346 | 52 | 0.07 |
| MMIBP0003 | 0.074 | 54 | 0.04 | 0.889 | 54 | 0.04 | 0.269 | 52 | 0.06 |
| MMIBP0006 | 0.389 | 54 | 0.07 | 0.37 | 54 | 0.07 | 0.308 | 52 | 0.06 |
| MMIBP0007 | 0.741 | 54 | 0.06 | 0.019 | 52 | 0.02 | 0.64 | 50 | 0.07 |
| MMIBP0008 | 0.296 | 54 | 0.06 | 0.352 | 54 | 0.06 | 0.558 | 52 | 0.07 |
| MMIBP0009 | 0.685 | 54 | 0.06 | 0.759 | 54 | 0.06 | 0.5 | 52 | 0.07 |
| MMIBP0010 | 0.212 | 52 | 0.06 | 0.963 | 54 | 0.03 | 0.604 | 48 | 0.07 |
| MMIBP0016 | 0.022 | 46 | 0.02 | 0.421 | 38 | 0.08 | 0.391 | 46 | 0.07 |
| MMIBP0017 | 0.074 | 54 | 0.04 | 0.712 | 52 | 0.06 | 0.173 | 52 | 0.05 |
| MMIBP0018 | 0.778 | 54 | 0.06 | 0.5 | 54 | 0.07 | 0.865 | 52 | 0.05 |
| MMIBP0019 | 0.667 | 54 | 0.06 | 0.885 | 52 | 0.04 | 0.692 | 52 | 0.06 |
| MMIBP0020 | 0.574 | 54 | 0.07 | 0.981 | 54 | 0.02 | 0.923 | 52 | 0.04 |
| MMIBP0021 | 0.75 | 52 | 0.06 | 0.963 | 54 | 0.03 | 0.635 | 52 | 0.07 |
| MMIBP0026 | 0.759 | 54 | 0.06 | 0.204 | 54 | 0.05 | 0.865 | 52 | 0.05 |
| MMIBP0027 | 0.409 | 44 | 0.07 | 0.833 | 48 | 0.05 | 0.5 | 46 | 0.07 |
| MMIBP0029 | 0 | 54 | 0 | 0.741 | 54 | 0.06 | 0.269 | 52 | 0.06 |
| MMIBP0031 | 0.222 | 54 | 0.06 | 0.648 | 54 | 0.06 | 0.327 | 52 | 0.07 |
| MMIBP0032 | 0.148 | 54 | 0.05 | 0.593 | 54 | 0.07 | 0.192 | 52 | 0.05 |
| MMIBP0034 | 0.295 | 44 | 0.07 | 0.021 | 48 | 0.02 | 0.619 | 42 | 0.07 |
| MMIBP0036 | 0.286 | 42 | 0.07 | 0.825 | 40 | 0.06 | 0.354 | 48 | 0.07 |
| MMIBP0038 | 0.192 | 52 | 0.05 | 0.519 | 54 | 0.07 | 0.404 | 52 | 0.07 |
| MMIBP0040 | 0.457 | 46 | 0.07 | 0.136 | 44 | 0.05 | 0.75 | 48 | 0.06 |
| MMIBP0041 | 0.654 | 52 | 0.07 | 0.135 | 52 | 0.05 | 0.346 | 52 | 0.07 |
| MMIBP0047 | 0.636 | 44 | 0.07 | 0.891 | 46 | 0.05 | 0.587 | 46 | 0.07 |
| MMIBP0048 | 0.56 | 50 | 0.07 | 0.94 | 50 | 0.03 | 0.68 | 50 | 0.07 |
| MMIBP0049 | 0.62 | 50 | 0.07 | 1 | 50 | 0 | 0.565 | 46 | 0.07 |
| MMIBP0051 | 0.917 | 48 | 0.04 | 0.146 | 48 | 0.05 | 0.826 | 46 | 0.06 |
| MMIBP0053 | 0.458 | 48 | 0.07 | 0.479 | 48 | 0.07 | 0.761 | 46 | 0.06 |
| MMIBP0054 | 0.605 | 38 | 0.08 | 0.022 | 46 | 0.02 | 0.705 | 44 | 0.07 |
| MMIBP0056 | 0.542 | 48 | 0.07 | 0.062 | 48 | 0.03 | 0.413 | 46 | 0.07 |
| MMIBP0057 | 1 | 48 | 0 | 0.083 | 48 | 0.04 | 1 | 46 | 0 |
| MMIBP0058 | 0.167 | 48 | 0.05 | 0.646 | 48 | 0.07 | 0.391 | 46 | 0.07 |
| MMIBP0060 | 0.083 | 48 | 0.04 | 1 | 46 | 0 | 0.304 | 46 | 0.07 |
| MMIBP0061 | 0.068 | 44 | 0.04 | 0.896 | 48 | 0.04 | 0.2 | 40 | 0.06 |
| MMIBP0063 | 0.413 | 46 | 0.07 | 0.146 | 48 | 0.05 | 0.609 | 46 | 0.07 |
| MMIBP0066 | 0.682 | 44 | 0.07 | 0.109 | 46 | 0.05 | 0.667 | 42 | 0.07 |
| MMIBP0067 | 0.542 | 48 | 0.07 | 0.812 | 48 | 0.06 | 0.304 | 46 | 0.07 |
| MMIBP0068 | 0.326 | 46 | 0.07 | 0.978 | 46 | 0.02 | 0.841 | 44 | 0.06 |
| MMIBP0070 | 0.146 | 48 | 0.05 | 0.167 | 48 | 0.05 | 0.152 | 46 | 0.05 |
| MMIBP0071 | 0.333 | 48 | 0.07 | 0.562 | 48 | 0.07 | 0.325 | 40 | 0.07 |
| MMIBP0074 | 0.85 | 40 | 0.06 | 0.974 | 38 | 0.03 | 0.556 | 36 | 0.08 |
| MMIBP0078 | 0.062 | 48 | 0.03 | 0.667 | 48 | 0.07 | 0.217 | 46 | 0.06 |
| MMIBP0079 | 0.13 | 46 | 0.05 | 0.458 | 48 | 0.07 | 0.619 | 42 | 0.07 |
| MMIBP0080 | 0.208 | 48 | 0.06 | 0.891 | 46 | 0.05 | 0.087 | 46 | 0.04 |
| MMIBP0082 | 0.646 | 48 | 0.07 | 0.958 | 48 | 0.03 | 0.625 | 40 | 0.08 |
| MMIBP0083 | 0.417 | 48 | 0.07 | 0.708 | 48 | 0.07 | 0.739 | 46 | 0.06 |
| MMIBP0084 | 0.725 | 40 | 0.07 | 0.717 | 46 | 0.07 | 0.325 | 40 | 0.07 |
| MMIBP0085 | 0.938 | 48 | 0.03 | 0.125 | 48 | 0.05 | 0.935 | 46 | 0.04 |
| MMIBP0087 | 0.568 | 44 | 0.07 | 0.104 | 48 | 0.04 | 0.37 | 46 | 0.07 |
| MMIBP0090 | 0.979 | 48 | 0.02 | 0.167 | 48 | 0.05 | 0.457 | 46 | 0.07 |
| MMIBP0093 | 0.604 | 48 | 0.07 | 0.413 | 46 | 0.07 | 0.5 | 44 | 0.08 |
| MMIBP0094 | 0.542 | 48 | 0.07 | 1 | 48 | 0 | 0.696 | 46 | 0.07 |
| MMIBP0095 | 0.708 | 48 | 0.07 | 0.667 | 48 | 0.07 | 0.674 | 46 | 0.07 |
| MMIBP0100 | 0.929 | 42 | 0.04 | 0.065 | 46 | 0.04 | 0.848 | 46 | 0.05 |
| MMIBP0102 | 0.25 | 48 | 0.06 | 0.109 | 46 | 0.05 | 0.432 | 44 | 0.07 |
| MMIBP0103 | 0.375 | 48 | 0.07 | 0.5 | 48 | 0.07 | 0.152 | 46 | 0.05 |
| MMIBP0109 | 0.292 | 48 | 0.07 | 0.375 | 48 | 0.07 | 0.152 | 46 | 0.05 |
| MMIBP0112 | 0.5 | 48 | 0.07 | 0.917 | 48 | 0.04 | 0.609 | 46 | 0.07 |
| MMIBP0113 | 0.575 | 40 | 0.08 | 0.833 | 36 | 0.06 | 0.711 | 38 | 0.07 |
| MMIBP0116 | 0.239 | 46 | 0.06 | 0.146 | 48 | 0.05 | 0.37 | 46 | 0.07 |
| MMIBP0117 | 0.333 | 48 | 0.07 | 0.295 | 44 | 0.07 | 0.37 | 46 | 0.07 |
| MMIBP0119 | 0.565 | 46 | 0.07 | 0.957 | 46 | 0.03 | 0.522 | 46 | 0.07 |
| MMIBP0120 | 0.083 | 48 | 0.04 | 0.833 | 48 | 0.05 | 0.31 | 42 | 0.07 |
| MMIBP0121 | 0.69 | 42 | 0.07 | 0.208 | 48 | 0.06 | 0.238 | 42 | 0.07 |
| MMIBP0123 | 0.476 | 42 | 0.08 | 0.146 | 48 | 0.05 | 0.875 | 40 | 0.05 |
| MMIBP0124 | 0.458 | 48 | 0.07 | 0.979 | 48 | 0.02 | 0.795 | 44 | 0.06 |
| MMIBP0125 | 0.667 | 48 | 0.07 | 0.891 | 46 | 0.05 | 0.452 | 42 | 0.08 |
| MMIBP0127 | 0.958 | 48 | 0.03 | 0.917 | 48 | 0.04 | 0.957 | 46 | 0.03 |
| MMIBP0128 | 0.176 | 34 | 0.07 | 0.825 | 40 | 0.06 | 0.333 | 42 | 0.07 |
| MMIBP0130 | 0.609 | 46 | 0.07 | 0.729 | 48 | 0.06 | 0.523 | 44 | 0.08 |
| MMIBP0131 | 0.289 | 38 | 0.07 | 0.864 | 44 | 0.05 | 0.524 | 42 | 0.08 |
| MMIBP0132 | 0.917 | 48 | 0.04 | 0.958 | 48 | 0.03 | 0.69 | 42 | 0.07 |
| MMIBP0133 | 0.675 | 40 | 0.07 | 0.2 | 40 | 0.06 | 0.344 | 32 | 0.08 |
| MMIBP0134 | 0.457 | 46 | 0.07 | 0.196 | 46 | 0.06 | 0.184 | 38 | 0.06 |
| MMIBP0135 | 0.368 | 38 | 0.08 | 0.977 | 44 | 0.02 | 0.633 | 30 | 0.09 |
| MMIBP0138 | 0.35 | 40 | 0.08 | 0 | 48 | 0 | 0.682 | 44 | 0.07 |
| MMIBP0139 | 0.595 | 42 | 0.08 | 0.708 | 48 | 0.07 | 0.553 | 38 | 0.08 |
| MMIBP0140 | 0.896 | 48 | 0.04 | 0.771 | 48 | 0.06 | 0.773 | 44 | 0.06 |
| MMIBP0141 | 0.708 | 48 | 0.07 | 0.896 | 48 | 0.04 | 0.457 | 46 | 0.07 |
| MMIBP0142 | 0.25 | 48 | 0.06 | 0.812 | 48 | 0.06 | 0.109 | 46 | 0.05 |
| MMIBP0144 | 0.417 | 48 | 0.07 | 0.896 | 48 | 0.04 | 0.674 | 46 | 0.07 |
| MMIBP0147 | 0.542 | 48 | 0.07 | 1 | 46 | 0 | 0.804 | 46 | 0.06 |
| MMIBP0148 | 0.696 | 46 | 0.07 | 1 | 46 | 0 | 0.571 | 42 | 0.08 |
| MMIBP0149 | 0.25 | 48 | 0.06 | 0.804 | 46 | 0.06 | 0.886 | 44 | 0.05 |
| MMIBP0150 | 0.023 | 44 | 0.02 | 0.667 | 42 | 0.07 | 0.525 | 40 | 0.08 |
| | Gelbvieh | No. of | | Hereford | No. of | | Holstein | No. of | |
| Alias | G Freq | Gametes | SE | G Freq | Gametes | SE | G Freq | Gametes | SE |
| MMIBP0001 | 0.615 | 52 | 0.07 | 0.308 | 52 | 0.06 | 0.4 | 40 | 0.08 |
| MMIBP0002 | 0.25 | 52 | 0.06 | 0.154 | 52 | 0.05 | 0.45 | 40 | 0.08 |
| MMIBP0003 | 0.019 | 52 | 0.02 | 0.058 | 52 | 0.03 | 0.25 | 40 | 0.07 |
| MMIBP0006 | 0.365 | 52 | 0.07 | 0.212 | 52 | 0.06 | 0.312 | 16 | 0.12 |
| MMIBP0007 | 0.682 | 44 | 0.07 | 0.673 | 52 | 0.07 | 0.706 | 34 | 0.08 |
| MMIBP0008 | 0.808 | 52 | 0.05 | 0.462 | 52 | 0.07 | 0.425 | 40 | 0.08 |
| MMIBP0009 | 0.269 | 52 | 0.06 | 0.577 | 52 | 0.07 | 0.3 | 40 | 0.07 |
| MMIBP0010 | 0.48 | 50 | 0.07 | 0.375 | 48 | 0.07 | 0.6 | 40 | 0.08 |
| MMIBP0016 | 0.262 | 42 | 0.07 | 0.12 | 50 | 0.05 | 0.421 | 38 | 0.08 |
| MMIBP0017 | 0.404 | 52 | 0.07 | 0 | 52 | 0 | 0.175 | 40 | 0.06 |
| MMIBP0018 | 0.904 | 52 | 0.04 | 0.712 | 52 | 0.06 | 0.675 | 40 | 0.07 |
| MMIBP0019 | 0.75 | 44 | 0.07 | 0.308 | 52 | 0.06 | 0.553 | 38 | 0.08 |
| MMIBP0020 | 0.846 | 52 | 0.05 | 0.788 | 52 | 0.06 | 0.7 | 40 | 0.07 |
| MMIBP0021 | 0.74 | 50 | 0.06 | 0.827 | 52 | 0.05 | | 0 | 0.00 |
| MMIBP0026 | 0.769 | 52 | 0.06 | 0.904 | 52 | 0.04 | 0.974 | 38 | 0.03 |
| MMIBP0027 | 0.591 | 44 | 0.07 | 0.5 | 48 | 0.07 | 0.5 | 38 | 0.08 |
| MMIBP0029 | 0.442 | 52 | 0.07 | 0.442 | 52 | 0.07 | 0.05 | 40 | 0.03 |
| MMIBP0031 | 0.154 | 52 | 0.05 | 0.135 | 52 | 0.05 | 0.1 | 40 | 0.05 |
| MMIBP0032 | 0.019 | 52 | 0.02 | 0.038 | 52 | 0.03 | 0.45 | 40 | 0.08 |
| MMIBP0034 | 0.556 | 36 | 0.08 | 0.783 | 46 | 0.06 | 0.4 | 40 | 0.08 |
| MMIBP0036 | 0.105 | 38 | 0.05 | 0.283 | 46 | 0.07 | 0.237 | 38 | 0.07 |
| MMIBP0038 | 0.519 | 52 | 0.07 | 0.481 | 52 | 0.07 | 0.225 | 40 | 0.07 |
| MMIBP0040 | 0.778 | 36 | 0.07 | 0.577 | 52 | 0.07 | 0.8 | 40 | 0.06 |
| MMIBP0041 | 0.42 | 50 | 0.07 | 0.635 | 52 | 0.07 | 0.225 | 40 | 0.07 |
| MMIBP0047 | 0.5 | 38 | 0.08 | 0.591 | 44 | 0.07 | | 0 | 0.00 |
| MMIBP0048 | 0.375 | 48 | 0.07 | 0.84 | 50 | 0.05 | 0.925 | 40 | 0.04 |
| MMIBP0049 | 0.625 | 40 | 0.08 | 0.712 | 52 | 0.06 | 0.35 | 40 | 0.08 |
| MMIBP0051 | 0.87 | 46 | 0.05 | 0.312 | 48 | 0.07 | 0.925 | 40 | 0.04 |
| MMIBP0053 | 0.739 | 46 | 0.06 | 0.729 | 48 | 0.06 | 0.4 | 40 | 0.08 |
| MMIBP0054 | 0.667 | 42 | 0.07 | 0.591 | 44 | 0.07 | 0.45 | 40 | 0.08 |
| MMIBP0056 | 0.674 | 46 | 0.07 | 0.326 | 46 | 0.07 | 0.605 | 38 | 0.08 |
| MMIBP0057 | 0.857 | 42 | 0.05 | 1 | 48 | 0 | 0.526 | 38 | 0.08 |
| MMIBP0058 | 0.227 | 44 | 0.06 | 0.042 | 48 | 0.03 | 0.105 | 38 | 0.05 |
| MMIBP0060 | 0.068 | 44 | 0.04 | 0.125 | 48 | 0.05 | 0.25 | 40 | 0.07 |
| MMIBP0061 | 0.522 | 46 | 0.07 | 0.227 | 44 | 0.06 | 0.316 | 38 | 0.08 |
| MMIBP0063 | 0.543 | 46 | 0.07 | 0.062 | 48 | 0.03 | 0.825 | 40 | 0.06 |
| MMIBP0066 | 0.553 | 38 | 0.08 | 0.562 | 48 | 0.07 | 0.825 | 40 | 0.06 |
| MMIBP0067 | 0.705 | 44 | 0.07 | 0.326 | 46 | 0.07 | 0.289 | 38 | 0.07 |
| MMIBP0068 | 0.636 | 44 | 0.07 | 0.739 | 46 | 0.06 | 0.5 | 38 | 0.08 |
| MMIBP0070 | 0.152 | 46 | 0.05 | 0.333 | 48 | 0.07 | 0.55 | 40 | 0.08 |
| MMIBP0071 | 0.5 | 42 | 0.08 | 0.833 | 48 | 0.05 | 0.275 | 40 | 0.07 |
| MMIBP0074 | 0.773 | 44 | 0.06 | 0.553 | 38 | 0.08 | 0.324 | 34 | 0.08 |
| MMIBP0078 | 0.152 | 46 | 0.05 | 0.208 | 48 | 0.06 | 0.2 | 40 | 0.06 |
| MMIBP0079 | 0.45 | 40 | 0.08 | 0.478 | 46 | 0.07 | 0.575 | 40 | 0.08 |
| MMIBP0080 | 0.136 | 44 | 0.05 | 0.083 | 48 | 0.04 | 0.5 | 40 | 0.08 |
| MMIBP0082 | 0.587 | 46 | 0.07 | 0.917 | 48 | 0.04 | 0.675 | 40 | 0.07 |
| MMIBP0083 | 0.717 | 46 | 0.07 | 0.413 | 46 | 0.07 | 0.825 | 40 | 0.06 |
| MMIBP0084 | 0.605 | 38 | 0.08 | 0.812 | 48 | 0.06 | 0.325 | 40 | 0.07 |
| MMIBP0085 | 0.87 | 46 | 0.05 | 0.646 | 48 | 0.07 | 0.775 | 40 | 0.07 |
| MMIBP0087 | 0.571 | 42 | 0.08 | 0.182 | 44 | 0.06 | 0.275 | 40 | 0.07 |
| MMIBP0090 | 0.587 | 46 | 0.07 | 0.646 | 48 | 0.07 | 0.875 | 40 | 0.05 |
| MMIBP0093 | 0.848 | 46 | 0.05 | 0.604 | 48 | 0.07 | 0.825 | 40 | 0.06 |
| MMIBP0094 | 0.37 | 46 | 0.07 | 0.729 | 48 | 0.06 | 0.55 | 40 | 0.08 |
| MMIBP0095 | 1 | 46 | 0 | 0.729 | 48 | 0.06 | 0.75 | 40 | 0.07 |
| MMIBP0100 | 0.955 | 44 | 0.03 | 0.896 | 48 | 0.04 | 0.625 | 40 | 0.08 |
| MMIBP0102 | 0.826 | 46 | 0.06 | 0.354 | 48 | 0.07 | 0.45 | 40 | 0.08 |
| MMIBP0103 | 0.304 | 46 | 0.07 | 0.521 | 48 | 0.07 | 0.325 | 40 | 0.07 |
| MMIBP0109 | 0.13 | 46 | 0.05 | 0.083 | 48 | 0.04 | 0.05 | 40 | 0.03 |
| MMIBP0112 | 0.614 | 44 | 0.07 | 0.413 | 46 | 0.07 | 0.425 | 40 | 0.08 |
| MMIBP0113 | 0.357 | 42 | 0.07 | 0.444 | 36 | 0.08 | 0.765 | 34 | 0.07 |
| MMIBP0116 | 0.348 | 46 | 0.07 | 0.354 | 48 | 0.07 | 0.425 | 40 | 0.08 |
| MMIBP0117 | 0.283 | 46 | 0.07 | 0.087 | 46 | 0.04 | 0.1 | 40 | 0.05 |
| MMIBP0119 | 0.69 | 42 | 0.07 | 0.478 | 46 | 0.07 | 0.875 | 40 | 0.05 |
| MMIBP0120 | 0.065 | 46 | 0.04 | 0.146 | 48 | 0.05 | 0.2 | 40 | 0.06 |
| MMIBP0121 | 0.477 | 44 | 0.08 | 0.5 | 44 | 0.08 | 0.474 | 38 | 0.08 |
| MMIBP0123 | 0.591 | 44 | 0.07 | 0.667 | 48 | 0.07 | 0.9 | 40 | 0.05 |
| MMIBP0124 | 0.63 | 46 | 0.07 | 0.833 | 48 | 0.05 | 0.875 | 40 | 0.05 |
| MMIBP0125 | 0.524 | 42 | 0.08 | 0.413 | 46 | 0.07 | 0.526 | 38 | 0.08 |
| MMIBP0127 | 0.696 | 46 | 0.07 | 0.979 | 48 | 0.02 | 0.658 | 38 | 0.08 |
| MMIBP0128 | 0.15 | 40 | 0.06 | 0.386 | 44 | 0.07 | 0.639 | 36 | 0.08 |
| MMIBP0130 | 0.13 | 46 | 0.05 | 0.25 | 48 | 0.06 | 0.132 | 38 | 0.05 |
| MMIBP0131 | 0.31 | 42 | 0.07 | 0.583 | 48 | 0.07 | 0.45 | 40 | 0.08 |
| MMIBP0132 | 0.652 | 46 | 0.07 | 0.562 | 48 | 0.07 | 0.3 | 40 | 0.07 |
| MMIBP0133 | 0.333 | 36 | 0.08 | 0.5 | 44 | 0.08 | 0.643 | 28 | 0.09 |
| MMIBP0134 | 0.643 | 42 | 0.07 | 0.042 | 48 | 0.03 | 0.184 | 38 | 0.06 |
| MMIBP0135 | 0.65 | 40 | 0.08 | 0.25 | 44 | 0.07 | 0.763 | 38 | 0.07 |
| MMIBP0138 | 0.6 | 40 | 0.08 | 0.625 | 48 | 0.07 | 0.575 | 40 | 0.08 |
| MMIBP0139 | 0.619 | 42 | 0.07 | 0.938 | 48 | 0.03 | 0.8 | 40 | 0.06 |
| MMIBP0140 | 0.568 | 44 | 0.07 | 0.5 | 48 | 0.07 | 0.632 | 38 | 0.08 |
| MMIBP0141 | 0.63 | 46 | 0.07 | 0.625 | 48 | 0.07 | 0.725 | 40 | 0.07 |
| MMIBP0142 | 0.174 | 46 | 0.06 | 0.062 | 48 | 0.03 | 0.175 | 40 | 0.06 |
| MMIBP0144 | 0.652 | 46 | 0.07 | 0.625 | 48 | 0.07 | 0.775 | 40 | 0.07 |
| MMIBP0147 | 0.523 | 44 | 0.08 | 0.812 | 48 | 0.06 | 0.8 | 40 | 0.06 |
| MMIBP0148 | 0.9 | 40 | 0.05 | 0.652 | 46 | 0.07 | 0.605 | 38 | 0.08 |
| MMIBP0149 | 0.848 | 46 | 0.05 | 0.458 | 48 | 0.07 | 0.825 | 40 | 0.06 |
| MMIBP0150 | 0.667 | 36 | 0.08 | 0.25 | 44 | 0.07 | 0.25 | 32 | 0.08 |
| | Limousin | No. of | | Simmental | No. of | | all | No. of | |
| Alias | G Freq | Gametes | SE | G Freq | Gametes | SE | G Freq | Gametes | SE |
| MMIBP0001 | 0.712 | 52 | 0.06 | 0.442 | 52 | 0.07 | 0.49 | 408 | 0.02 |
| MMIBP0002 | 0.212 | 52 | 0.06 | 0.288 | 52 | 0.06 | 0.341 | 408 | 0.02 |
| MMIBP0003 | 0.173 | 52 | 0.05 | 0.019 | 52 | 0.02 | 0.221 | 408 | 0.02 |
| MMIBP0006 | 0.558 | 52 | 0.07 | 0.192 | 52 | 0.05 | 0.341 | 384 | 0.02 |
| MMIBP0007 | 0.769 | 52 | 0.06 | 0.442 | 52 | 0.07 | 0.577 | 390 | 0.03 |
| MMIBP0008 | 0.75 | 52 | 0.06 | 0.423 | 52 | 0.07 | 0.51 | 408 | 0.02 |
| MMIBP0009 | 0.404 | 52 | 0.07 | 0.25 | 52 | 0.06 | 0.475 | 408 | 0.02 |
| MMIBP0010 | 0.62 | 50 | 0.07 | 0.44 | 50 | 0.07 | 0.538 | 392 | 0.03 |
| MMIBP0016 | 0.4 | 40 | 0.08 | 0.364 | 44 | 0.07 | 0.291 | 344 | 0.02 |
| MMIBP0017 | 0.077 | 52 | 0.04 | 0.077 | 52 | 0.04 | 0.212 | 406 | 0.02 |
| MMIBP0018 | 0.846 | 52 | 0.05 | 0.75 | 52 | 0.06 | 0.755 | 408 | 0.02 |
| MMIBP0019 | 0.64 | 50 | 0.07 | 0.692 | 52 | 0.06 | 0.65 | 394 | 0.02 |
| MMIBP0020 | 0.75 | 52 | 0.06 | 0.788 | 52 | 0.06 | 0.797 | 408 | 0.02 |
| MMIBP0021 | 0.385 | 52 | 0.07 | 0.808 | 52 | 0.05 | 0.731 | 364 | 0.02 |
| MMIBP0026 | 0.788 | 52 | 0.06 | 0.558 | 52 | 0.07 | 0.717 | 406 | 0.02 |
| MMIBP0027 | 0.386 | 44 | 0.07 | 0.409 | 44 | 0.07 | 0.52 | 356 | 0.03 |
| MMIBP0029 | 0.173 | 52 | 0.05 | 0.308 | 52 | 0.06 | 0.311 | 408 | 0.02 |
| MMIBP0031 | 0.231 | 52 | 0.06 | 0.019 | 52 | 0.02 | 0.235 | 408 | 0.02 |
| MMIBP0032 | 0.327 | 52 | 0.07 | 0.058 | 52 | 0.03 | 0.223 | 408 | 0.02 |
| MMIBP0034 | 0.525 | 40 | 0.08 | 0.69 | 42 | 0.07 | 0.479 | 338 | 0.03 |
| MMIBP0036 | 0.214 | 42 | 0.06 | 0.208 | 48 | 0.06 | 0.313 | 342 | 0.03 |
| MMIBP0038 | 0.212 | 52 | 0.06 | 0.577 | 52 | 0.07 | 0.397 | 406 | 0.02 |
| MMIBP0040 | 0.727 | 44 | 0.07 | 0.55 | 40 | 0.08 | 0.591 | 350 | 0.03 |
| MMIBP0041 | 0.635 | 52 | 0.07 | 0.308 | 52 | 0.06 | 0.425 | 402 | 0.02 |
| MMIBP0047 | 0.75 | 40 | 0.07 | 0.762 | 42 | 0.07 | 0.677 | 300 | 0.03 |
| MMIBP0048 | 0.353 | 34 | 0.08 | 0.5 | 48 | 0.07 | 0.654 | 370 | 0.02 |
| MMIBP0049 | 0.565 | 46 | 0.07 | 0.479 | 48 | 0.07 | 0.624 | 372 | 0.03 |
| MMIBP0051 | 0.63 | 46 | 0.07 | 0.812 | 48 | 0.06 | 0.673 | 370 | 0.02 |
| MMIBP0053 | 0.652 | 46 | 0.07 | 0.833 | 48 | 0.05 | 0.635 | 370 | 0.03 |
| MMIBP0054 | 0.591 | 44 | 0.07 | 0.63 | 46 | 0.07 | 0.529 | 344 | 0.03 |
| MMIBP0056 | 0.381 | 42 | 0.07 | 0.659 | 44 | 0.07 | 0.453 | 358 | 0.03 |
| MMIBP0057 | 0.935 | 46 | 0.04 | 0.913 | 46 | 0.04 | 0.793 | 362 | 0.02 |
| MMIBP0058 | 0.196 | 46 | 0.06 | 0.5 | 46 | 0.07 | 0.288 | 364 | 0.02 |
| MMIBP0060 | 0.25 | 44 | 0.07 | 0.091 | 44 | 0.04 | 0.272 | 360 | 0.02 |
| MMIBP0061 | 0.283 | 46 | 0.07 | 0.295 | 44 | 0.07 | 0.36 | 350 | 0.03 |
| MMIBP0063 | 0.452 | 42 | 0.08 | 0.568 | 44 | 0.07 | 0.442 | 360 | 0.03 |
| MMIBP0066 | 0.435 | 46 | 0.07 | 0.625 | 48 | 0.07 | 0.551 | 352 | 0.03 |
| MMIBP0067 | 0.182 | 44 | 0.06 | 0.565 | 46 | 0.07 | 0.472 | 360 | 0.03 |
| MMIBP0068 | 0.548 | 42 | 0.08 | 0.761 | 46 | 0.06 | 0.67 | 352 | 0.03 |
| MMIBP0070 | 0.196 | 46 | 0.06 | 0.229 | 48 | 0.06 | 0.235 | 370 | 0.02 |
| MMIBP0071 | 0.522 | 46 | 0.07 | 0.312 | 48 | 0.07 | 0.464 | 360 | 0.03 |
| MMIBP0074 | 0.65 | 40 | 0.08 | 0.636 | 44 | 0.07 | 0.672 | 314 | 0.03 |
| MMIBP0078 | 0.174 | 46 | 0.06 | 0.312 | 48 | 0.07 | 0.251 | 370 | 0.02 |
| MMIBP0079 | 0.63 | 46 | 0.07 | 0.543 | 46 | 0.07 | 0.483 | 354 | 0.03 |
| MMIBP0080 | 0.25 | 44 | 0.07 | 0.109 | 46 | 0.05 | 0.279 | 362 | 0.02 |
| MMIBP0082 | 0.804 | 46 | 0.06 | 0.477 | 44 | 0.08 | 0.717 | 360 | 0.02 |
| MMIBP0083 | 0.261 | 46 | 0.06 | 0.646 | 48 | 0.07 | 0.587 | 368 | 0.03 |
| MMIBP0084 | 0.587 | 46 | 0.07 | 0.714 | 42 | 0.07 | 0.609 | 340 | 0.03 |
| MMIBP0085 | 0.804 | 46 | 0.06 | 0.938 | 48 | 0.03 | 0.751 | 370 | 0.02 |
| MMIBP0087 | 0.348 | 46 | 0.07 | 0.391 | 46 | 0.07 | 0.348 | 356 | 0.03 |
| MMIBP0090 | 1 | 46 | 0 | 0.783 | 46 | 0.06 | 0.682 | 368 | 0.02 |
| MMIBP0093 | 0.848 | 46 | 0.05 | 0.667 | 48 | 0.07 | 0.661 | 366 | 0.02 |
| MMIBP0094 | 0.674 | 46 | 0.07 | 0.457 | 46 | 0.07 | 0.63 | 368 | 0.03 |
| MMIBP0095 | 0.727 | 44 | 0.07 | 0.854 | 48 | 0.05 | 0.764 | 368 | 0.02 |
| MMIBP0100 | 1 | 46 | 0 | 0.976 | 42 | 0.02 | 0.785 | 354 | 0.02 |
| MMIBP0102 | 0.348 | 46 | 0.07 | 0.542 | 48 | 0.07 | 0.413 | 366 | 0.03 |
| MMIBP0103 | 0.5 | 46 | 0.07 | 0.292 | 48 | 0.07 | 0.373 | 370 | 0.03 |
| MMIBP0109 | 0.565 | 46 | 0.07 | 0.196 | 46 | 0.06 | 0.234 | 368 | 0.02 |
| MMIBP0112 | 0.804 | 46 | 0.06 | 0.667 | 48 | 0.07 | 0.623 | 366 | 0.03 |
| MMIBP0113 | 0.238 | 42 | 0.07 | 0.405 | 42 | 0.08 | 0.529 | 310 | 0.03 |
| MMIBP0116 | 0.587 | 46 | 0.07 | 0.391 | 46 | 0.07 | 0.355 | 366 | 0.03 |
| MMIBP0117 | 0.261 | 46 | 0.06 | 0.364 | 44 | 0.07 | 0.264 | 360 | 0.02 |
| MMIBP0119 | 0.5 | 46 | 0.07 | 0.333 | 42 | 0.07 | 0.613 | 354 | 0.03 |
| MMIBP0120 | 0.065 | 46 | 0.04 | 0.104 | 48 | 0.04 | 0.227 | 366 | 0.02 |
| MMIBP0121 | 0.341 | 44 | 0.07 | 0.364 | 44 | 0.07 | 0.408 | 346 | 0.03 |
| MMIBP0123 | 0.571 | 42 | 0.08 | 0.705 | 44 | 0.07 | 0.606 | 348 | 0.03 |
| MMIBP0124 | 0.522 | 46 | 0.07 | 0.609 | 46 | 0.07 | 0.71 | 366 | 0.02 |
| MMIBP0125 | 0.55 | 40 | 0.08 | 0.341 | 44 | 0.07 | 0.549 | 346 | 0.03 |
| MMIBP0127 | 0.738 | 42 | 0.07 | 0.435 | 46 | 0.07 | 0.798 | 362 | 0.02 |
| MMIBP0128 | 0.095 | 42 | 0.05 | 0.265 | 34 | 0.08 | 0.359 | 312 | 0.03 |
| MMIBP0130 | 0.5 | 46 | 0.07 | 0.286 | 42 | 0.07 | 0.402 | 358 | 0.03 |
| MMIBP0131 | 0.636 | 44 | 0.07 | 0.619 | 42 | 0.07 | 0.541 | 340 | 0.03 |
| MMIBP0132 | 0.63 | 46 | 0.07 | 0.667 | 48 | 0.07 | 0.68 | 366 | 0.02 |
| MMIBP0133 | 0.5 | 38 | 0.08 | 0.548 | 42 | 0.08 | 0.467 | 300 | 0.03 |
| MMIBP0134 | 0.196 | 46 | 0.06 | 0.477 | 44 | 0.08 | 0.296 | 348 | 0.02 |
| MMIBP0135 | 0.618 | 34 | 0.08 | 0.667 | 36 | 0.08 | 0.615 | 304 | 0.03 |
| MMIBP0138 | 0.682 | 44 | 0.07 | 0.682 | 44 | 0.07 | 0.52 | 348 | 0.03 |
| MMIBP0139 | 0.609 | 46 | 0.07 | 0.571 | 42 | 0.08 | 0.679 | 346 | 0.03 |
| MMIBP0140 | 0.5 | 44 | 0.08 | 0.652 | 46 | 0.07 | 0.664 | 360 | 0.02 |
| MMIBP0141 | 0.591 | 44 | 0.07 | 0.478 | 46 | 0.07 | 0.639 | 366 | 0.03 |
| MMIBP0142 | 0.239 | 46 | 0.06 | 0.326 | 46 | 0.07 | 0.272 | 368 | 0.02 |
| MMIBP0144 | 0.587 | 46 | 0.07 | 0.652 | 46 | 0.07 | 0.658 | 368 | 0.02 |
| MMIBP0147 | 0.727 | 44 | 0.07 | 0.479 | 48 | 0.07 | 0.709 | 364 | 0.02 |
| MMIBP0148 | 0.727 | 44 | 0.07 | 0.9 | 40 | 0.05 | 0.757 | 342 | 0.02 |
| MMIBP0149 | 0.696 | 46 | 0.07 | 0.739 | 46 | 0.06 | 0.681 | 364 | 0.02 |
| MMIBP0150 | 0.4 | 40 | 0.08 | 0.778 | 36 | 0.07 | 0.436 | 314 | 0.03 |

Although the invention has been described with reference to the above example, it will be understood that modifications and variations are encompassed within the spirit and scope of the invention. Accordingly, the invention is limited only by the following claims.

What is claimed is:

1. A method to infer parentage of a bovine subject from a nucleic acid sample of the bovine subject, comprising identifying in the nucleic acid sample at least one nucleotide occurrence of at least one single nucleotide polymorphism (SNP) corresponding to the first nucleotide, or the complement thereof, in the 3′ position to any one of SEQ ID NOs: 261-390, thereby inferring the identity of the bovine subject.

2. The method of claim 1, wherein the nucleotide incorporated immediately proximal to the 3′ end of each primer is an extendible or non-extendible nucleotide.

3. The method of claim 2, wherein the non-extendible nucleotide is a ddNTP.

4. The method of claim 3, wherein the ddNTP is fluorescently or chemically labeled.

5. The method of claim 3, wherein the ddNTP is biotinylated.

6. The method of claim 1, wherein the target nucleic acid molecule is a DNA molecule.

7. The method of claim 6, wherein the DNA molecule is genomic DNA.

8. The method of claim 6, wherein the DNA molecule is double-stranded DNA.

9. The method of claim 6, wherein the DNA molecule is single-stranded DNA.

10. The method of claim 6, wherein the nucleic acid molecule is an RNA molecule.

11. A method to infer parentage of a bovine subject from a nucleic acid sample of the bovine subject, the method comprising:
a) contacting the nucleic acid sample with a pair of oligonucleotides that comprise a primer pair, wherein amplified target nucleic acid molecules are produced;
b) hybridizing at least one oligonucleotide primer selected from the group consisting of SEQ ID NOS: 261-390 to one or more amplified target nucleic acid molecules, wherein each oligonucleotide primer is complementary to a specific and unique region of each target nucleic acid molecule such that the 3′ end of each primer is immediately proximal to a specific and unique target nucleotide of interest;
c) extending each oligonucleotide with a template-dependent polymerase; and
d) determining the identity of each nucleotide of interest by determining, for each extension primer employed, the identity of the nucleotide immediately proximal to the 3′ end of each primer.

12. The method of claim 11, wherein the primer pair is any of the forward and reverse primer pairs listed in Table 1.

13. The method of claim 11, wherein a first primer of the primer pair is selected from SEQ ID NOS:1-130 and the second primer of the primer pair is selected from SEQ ID NOS:131-260.

14. An isolated oligonucleotide comprising any one of SEQ ID NOS: 261-390, wherein each oligonucleotide further includes one additional nucleotide positioned immediately proximal to the 3′ end of each oligonucleotide, wherein the oligonucleotide specifically hybridizes to a nucleic acid sequence derived from a bovine animal.

15. The complement of the oligonucleotide of claim 14.

16. Isolated oligonucleotide marker sets as set forth in Table 1.

17. An isolated oligonucleotide marker set selected from from the group consisting of marker set MMIBP0001 through MMIBP0150 of Table 1.

18. A method for identifying the parentage of a bovine test subject, the method comprising:
a) obtaining a nucleic acid sample from the test subject by a method comprising identifying in the nucleic acid sample at least one nucleotide occurrence of at least one single nucleotide polymorphism (SNP) corresponding to the first nucleotide, or the complement thereof, in the 3′ position to any one of SEQ ID NOs: 261-390; and
b) repeating a) for additional subjects;
c) determining the allele frequency corresponding to each SNP identified; and
d) comparing the allele frequency of the test subject with each additional subject.

19. The method of claim 18, wherein the additional bovine subjects can be the same breed or a different breed as the test subject.

20. A kit for determining nucleotide occurrences of bovine SNPs, the kit comprising an oligonucleotide probe, primer, or primer pair, or combinations thereof, for identifying the nucleotide occurrence of at least one bovine single nucleotide polymorphism (SNP) corresponding to the first nucleotide, or the complement thereof, in the 3′ position to any one of SEQ ID NOs: 261-390, wherein the SNP is associated with parentage.

21. A kit for determining nucleotide occurrences of bovine SNPs, the kit comprising at least one oligonucleotide marker set as set forth in Table 1.

22. The kit of claim 21, wherein the marker set is selected from the group consisting of marker set MMIBP0001 through MMIBP0150 of Table 1.

23. The kit of claims 20, 21 or 22, further comprising one or more detectable labels.

24. A database comprising allele frequencies generated by identifying, in a nucleic acid sample derived from a bovine subject, the single nucleotide polymorphism (SNP) corresponding to the first nucleotide, or the complement thereof, in the 3′ position to each of the oligonucleotides set forth in SEQ ID NOS: 261-390.

25. A database comprising allele frequencies generated by identifying, in a nucleic acid sample derived from a bovine subject, the single nucleotide polymorphisms (SNP) identified by the marker sets MMIBP0001 through MMIBP0150 of Table 1.

26. A database comprising the allele frequencies set forth in Table 2.

27. A computer-based method for identifying the parentage of a bovine subject, the method comprising:
a) obtaining a nucleic acid sample from the bovine subject;
b) identifying in the nucleic acid sample at least one nucleotide occurrence of at least one single nucleotide polymorphism (SNP) corresponding to the first nucleotide, or the complement thereof, in the 3′ position to any one of SEQ ID NOs: 261-390;
c) searching a database comprising allele frequencies generated by the marker sets of claim 16;
d) retrieving the information from database;
e) optionally storing the information in a memory location associated with a user such that the information may be subsequently accessed and viewed by the user; and
f) identifying the parentage of a bovine subject.


SEQUENCE LISTING (390 sequences)

SEQ ID NO: 1 - DNA, 25 bp, Artificial sequence
  Primer
tttacctacctcataaaaatgctct

SEQ ID NO: 2 - DNA, 18 bp, Artificial sequence
  Primer
tcccgcatccccacttct

SEQ ID NO: 3 - DNA, 19 bp, Artificial sequence
  Primer
tgccagtctgaagaaacca

SEQ ID NO: 4 - DNA, 24 bp, Artificial sequence
  Primer
tgtttgtatcttccaaatttcata

SEQ ID NO: 5 - DNA, 22 bp, Artificial sequence
  Primer
ttatgtaatcccagggatgttg

SEQ ID NO: 6 - DNA, 27 bp, Artificial sequence
  Primer
ttttagtctgagtgtaaataacttggg

SEQ ID NO: 7 - DNA, 22 bp, Artificial sequence
  Primer
aaataactccgtgaatgtgtgg

SEQ ID NO: 8 - DNA, 20 bp, Artificial sequence
  Primer
ttaaagtgtggagcctggag

SEQ ID NO: 9 - DNA, 25 bp, Artificial sequence
  Primer
aaattgactractgttttttgtcac

SEQ ID NO: 10 - DNA, 22 bp, Artificial sequence
  Primer
aagtgcaaggtcttaaccactg

SEQ ID NO: 11 - DNA, 31 bp, Artificial sequence
  Primer
attattatcttgttttactttggtaagagac

SEQ ID NO: 12 - DNA, 18 bp, Artificial sequence
  Primer
tcaggtgattgccgttgt

SEQ ID NO: 13 - DNA, 23 bp, Artificial sequence
  Primer
atttcctacttttgcattaccca

SEQ ID NO: 14 - DNA, 24 bp, Artificial sequence
  Primer
aaatgatagttgtggcagtatata

SEQ ID NO: 15 - DNA, 24 bp, Artificial sequence
  Primer
aatctgtttctgagcttgttcttg

SEQ ID NO: 16 - DNA, 28 bp, Artificial sequence
  Primer
cttaaaacatgtatttgtctttctactt

SEQ ID NO: 17 - DNA, 25 bp, Artificial sequence
  Primer
acacactgcaattaatagaggattc

SEQ ID NO: 18 - DNA, 25 bp, Artificial sequence
  Primer
aacaataaaatgtcatgtaygtcaa

SEQ ID NO: 19 - DNA, 21 bp, Artificial sequence
  Primer
ttctctctggactctgtgcag

SEQ ID NO: 20 - DNA, 20 bp, Artificial sequence
  Primer
aaagaaagggtgagggtgaa

SEQ ID NO: 21 - DNA, 21 bp, Artificial sequence
  Primer
ataagatgctggctgagacct

SEQ ID NO: 22 - DNA, 18 bp, Artificial sequence
  Primer
agaccgtcaggagctgag

SEQ ID NO: 23 - DNA, 21 bp, Artificial sequence
  Primer
acctgtttgccatcttctttc

SEQ ID NO: 24 - DNA, 22 bp, Artificial sequence
  Primer
ttttggcatacatcaacttgaa

SEQ ID NO: 25 - DNA, 26 bp, Artificial sequence
  Primer
cccaaactaaaaatgatttaagaaac

SEQ ID NO: 26 - DNA, 19 bp, Artificial sequence
  Primer
tctgcaatctggcattgag

SEQ ID NO: 27 - DNA, 23 bp, Artificial sequence
  Primer
atatccatccctttctcatctgt

SEQ ID NO: 28 - DNA, 21 bp, Artificial sequence
  Primer
atctttcagtcatgccagatc

SEQ ID NO: 29 - DNA, 24 bp, Artificial sequence
  Primer
ggcagtcactgactctgtaatagg

SEQ ID NO: 30 - DNA, 18 bp, Artificial sequence
  Primer
ctgccctcttctccaacc

SEQ ID NO: 31 - DNA, 21 bp, Artificial sequence
  Primer
acgaaaagtgctttgtgaaaa

SEQ ID NO: 32 - DNA, 28 bp, Artificial sequence
  Primer
acttgttgaaaaactctaaaggtaaatt

SEQ ID NO: 33 - DNA, 19 bp, Artificial sequence
  Primer
tgaatgagcaaaggtcagg

SEQ ID NO: 34 - DNA, 18 bp, Artificial sequence
  Primer
tccacctgcttcctctgg

SEQ ID NO: 35 - DNA, 26 bp, Artificial sequence
  Primer
aaactgaaggttctttttggtatagg

SEQ ID NO: 36 - DNA, 28 bp, Artificial sequence
  Primer
gatatagaggacttttacgagtttcatt

SEQ ID NO: 37 - DNA, 32 bp, Artificial sequence
  Primer
gtaatacttcatgtagatttttaaactttgag

SEQ ID NO: 38 - DNA, 27 bp, Artificial sequence
  Primer
gaggtatatgatgaaaacagcttagag

SEQ ID NO: 39 - DNA, 29 bp, Artificial sequence
  Primer
aaaagtaacttactgaaccaatattgaca

SEQ ID NO: 40 - DNA, 18 bp, Artificial sequence
  Primer
attggggcatgaacactg

SEQ ID NO: 41 - DNA, 18 bp, Artificial sequence
  Primer
actccgcatccctggacc

SEQ ID NO: 42 - DNA, 18 bp, Artificial sequence
  Primer
ctgggactgaaaggggat

SEQ ID NO: 43 - DNA, 18 bp, Artificial sequence
  Primer
agcagagtcccagggcag

SEQ ID NO: 44 - DNA, 24 bp, Artificial sequence
  Primer
ttgaacaagaggatgatattctgc

SEQ ID NO: 45 - DNA, 22 bp, Artificial sequence
  Primer
aaggtggaacaaaagcagtatt

SEQ ID NO: 46 - DNA, 20 bp, Artificial sequence
  Primer
cttttccttttggtcctctg

SEQ ID NO: 47 - DNA, 28 bp, Artificial sequence
  Primer
aggagatatatgtttgaaatttaggtca

SEQ ID NO: 48 - DNA, 27 bp, Artificial sequence
  Primer
aaatttaaacagaattcctacttagca

SEQ ID NO: 49 - DNA, 19 bp, Artificial sequence
  Primer
tgaaccagattccacctca

SEQ ID NO: 50 - DNA, 19 bp, Artificial sequence
  Primer
agaaactggaactgctcga

SEQ ID NO: 51 - DNA, 26 bp, Artificial sequence
  Primer
attccagaagtctgttttaaaatgtc

SEQ ID NO: 52 - DNA, 27 bp, Artificial sequence
  Primer
ttatttactgtttgcttctgttatttc

SEQ ID NO: 53 - DNA, 25 bp, Artificial sequence
  Primer
aaayaagatgaccattaggttgatg

SEQ ID NO: 54 - DNA, 19 bp, Artificial sequence
  Primer
acgagtgaatgaagggaac

SEQ ID NO: 55 - DNA, 32 bp, Artificial sequence
  Primer
aatatgaaagttctgtaagtataaaacagtgt

SEQ ID NO: 56 - DNA, 26 bp, Artificial sequence
  Primer
gcagcccagtataataataatagctc

SEQ ID NO: 57 - DNA, 19 bp, Artificial sequence
  Primer
tcaaatgcctaccctggtg

SEQ ID NO: 58 - DNA, 25 bp, Artificial sequence
  Primer
aatgtttggctactagagtgagtga

SEQ ID NO: 59 - DNA, 27 bp, Artificial sequence
  Primer
atatattgccaatartgatcactttca

SEQ ID NO: 60 - DNA, 24 bp, Artificial sequence
  Primer
atttttgaattagagcctttgaca

SEQ ID NO: 61 - DNA, 20 bp, Artificial sequence
  Primer
ttcatcccctaaaaaggagc

SEQ ID NO: 62 - DNA, 18 bp, Artificial sequence
  Primer
actgcaaatggcaaggaa

SEQ ID NO: 63 - DNA, 24 bp, Artificial sequence
  Primer
aaagacacttccacctagttctcc

SEQ ID NO: 64 - DNA, 22 bp, Artificial sequence
  Primer
ttttctgaaataattcccacca

SEQ ID NO: 65 - DNA, 26 bp, Artificial sequence
  Primer
tattaagaaactactcgcagatgtga

SEQ ID NO: 66 - DNA, 28 bp, Artificial sequence
  Primer
ttcttattttagacactatctcaagcat

SEQ ID NO: 67 - DNA, 22 bp, Artificial sequence
  Primer
tacgatgtgttcacatagccat

SEQ ID NO: 68 - DNA, 18 bp, Artificial sequence
  Primer
cccaaagggtaaaatggc

SEQ ID NO: 69 - DNA, 20 bp, Artificial sequence
  Primer
attcaatggcactaaggcag

SEQ ID NO: 70 - DNA, 23 bp, Artificial sequence
  Primer
aaaacagaacaactacttgcctg

SEQ ID NO: 71 - DNA, 21 bp, Artificial sequence
  Primer
ctgcttgctgttcagataacg

SEQ ID NO: 72 - DNA, 18 bp, Artificial sequence
  Primer
aacggcttggcaaaggta

SEQ ID NO: 73 - DNA, 25 bp, Artificial sequence
  Primer
tattatagcttctttcagagctggg

SEQ ID NO: 74 - DNA, 27 bp, Artificial sequence
  Primer
tacatatcttatatattcaggatccct

SEQ ID NO: 75 - DNA, 26 bp, Artificial sequence
  Primer
atcttcctaatgccacttttatttat

SEQ ID NO: 76 - DNA, 20 bp, Artificial sequence
  Primer
aatggttgactgccatgatg

SEQ ID NO: 77 - DNA, 23 bp, Artificial sequence
  Primer
accaaaaaaaytcacaataagcc

SEQ ID NO: 78 - DNA, 24 bp, Artificial sequence
  Primer
atgaatgtttgacttttgaattgt

SEQ ID NO: 79 - DNA, 23 bp, Artificial sequence
  Primer
acttggtgactgaacaacaaaat

SEQ ID NO: 80 - DNA, 18 bp, Artificial sequence
  Primer
tttctcatccctccccct

SEQ ID NO: 81 - DNA, 18 bp, Artificial sequence
  Primer
tgtgctccgtgttccaga

SEQ ID NO: 82 - DNA, 19 bp, Artificial sequence
  Primer
ctgaactgagaaggaggga

SEQ ID NO: 83 - DNA, 25 bp, Artificial sequence
  Primer
atatgttgatgtcatagtacacccc

SEQ ID NO: 84 - DNA, 21 bp, Artificial sequence
  Primer
ttaggttaccaggtgtagccc

SEQ ID NO: 85 - DNA, 19 bp, Artificial sequence
  Primer
tggcacagcatcttgtctc

SEQ ID NO: 86 - DNA, 25 bp, Artificial sequence
  Primer
catttaagttgtccacctatgaagt

SEQ ID NO: 87 - DNA, 30 bp, Artificial sequence
  Primer
aaattataaaaaagcattctagtcagagtc

SEQ ID NO: 88 - DNA, 26 bp, Artificial sequence
  Primer
ttaattaattacaaaatgcagctgtg

SEQ ID NO: 89 - DNA, 25 bp, Artificial sequence
  Primer
tgaatttctacctcaatttctagcc

SEQ ID NO: 90 - DNA, 18 bp, Artificial sequence
  Primer
atggtcaccggacacagc

SEQ ID NO: 91 - DNA, 24 bp, Artificial sequence
  Primer
aatacatcaaccagcttaggtgtt

SEQ ID NO: 92 - DNA, 29 bp, Artificial sequence
  Primer
ctgaaattattcacatattcactataagc

SEQ ID NO: 93 - DNA, 21 bp, Artificial sequence
  Primer
aacatgatccccctcttactg

SEQ ID NO: 94 - DNA, 22 bp, Artificial sequence
  Primer
tgtccaagtctctatgtttctg

SEQ ID NO: 95 - DNA, 24 bp, Artificial sequence
  Primer
acacataacagatttcctaatttg

SEQ ID NO: 96 - DNA, 26 bp, Artificial sequence
  Primer
tttttagaaatcaataagacaggtga

SEQ ID NO: 97 - DNA, 26 bp, Artificial sequence
  Primer
acaaaataatgcaaatataatcctcc

SEQ ID NO: 98 - DNA, 30 bp, Artificial sequence
  Primer
ttattaactgtctattacatgttagggtaa

SEQ ID NO: 99 - DNA, 26 bp, Artificial sequence
  Primer
tatcaatgtccttttttacaactttc

SEQ ID NO: 100 - DNA, 22 bp, Artificial sequence
  Primer
aattgaagaggaagaaaattgg

SEQ ID NO: 101 - DNA, 21 bp, Artificial sequence
  Primer
aaatcatgagttggggtcttc

SEQ ID NO: 102 - DNA, 21 bp, Artificial sequence
  Primer
gaagttaactccaaagcagat

SEQ ID NO: 103 - DNA, 32 bp, Artificial sequence
  Primer
tctacactatcaaaattatcatattttacctc

SEQ ID NO: 104 - DNA, 22 bp, Artificial sequence
  Primer
ttaaaaggaaagtctgctgctg

SEQ ID NO: 105 - DNA, 22 bp, Artificial sequence
  Primer
ttgctaagtctttgggaatctc

SEQ ID NO: 106 - DNA, 20 bp, Artificial sequence
  Primer
tcagaaagggcatacatcaa

SEQ ID NO: 107 - DNA, 19 bp, Artificial sequence
  Primer
aggaagatgagtcaccggt

SEQ ID NO: 108 - DNA, 19 bp, Artificial sequence
  Primer
agagaatcaggcacaaggc

SEQ ID NO: 109 - DNA, 19 bp, Artificial sequence
  Primer
actgacacctccatccatc

SEQ ID NO: 110 - DNA, 21 bp, Artificial sequence
  Primer
ctccataatgaacaaaaccct

SEQ ID NO: 111 - DNA, 26 bp, Artificial sequence
  Primer
aaaaggaagtcttattcaggtgatag

SEQ ID NO: 112 - DNA, 20 bp, Artificial sequence
  Primer
taccacagaggaaactttgg

SEQ ID NO: 113 - DNA, 25 bp, Artificial sequence
  Primer
tatattgaatttaaatggctcacca

SEQ ID NO: 114 - DNA, 19 bp, Artificial sequence
  Primer
tagagaaaagtggcgcagc

SEQ ID NO: 115 - DNA, 27 bp, Artificial sequence
  Primer
taagaaaggttaattaggaagagaagc

SEQ ID NO: 116 - DNA, 31 bp, Artificial sequence
  Primer
tcctactaggtgactagtatatctgtacatg

SEQ ID NO: 117 - DNA, 22 bp, Artificial sequence
  Primer
aatttcctttttatccaatgcc

SEQ ID NO: 118 - DNA, 19 bp, Artificial sequence
  Primer
acacatcacacagcctccc

SEQ ID NO: 119 - DNA, 22 bp, Artificial sequence
  Primer
aaaatttggtgctttgatcact

SEQ ID NO: 120 - DNA, 26 bp, Artificial sequence
  Primer
tagatgtggtaaacaacgaagagtaa

SEQ ID NO: 121 - DNA, 18 bp, Artificial sequence
  Primer
tgccacatgcgaggacta

SEQ ID NO: 122 - DNA, 22 bp, Artificial sequence
  Primer
tttgaagaaaaacattactggg

SEQ ID NO: 123 - DNA, 26 bp, Artificial sequence
  Primer
atttcaaacagcacagaagttatagg

SEQ ID NO: 124 - DNA, 24 bp, Artificial sequence
  Primer
tagctgagtccagtctaaactcct

SEQ ID NO: 125 - DNA, 23 bp, Artificial sequence
  Primer
tgtgatctatttggtttgatgag

SEQ ID NO: 126 - DNA, 18 bp, Artificial sequence
  Primer
agaggacagggggacctg

SEQ ID NO: 127 - DNA, 18 bp, Artificial sequence
  Primer
aactgcagtgcttgaggg

SEQ ID NO: 128 - DNA, 18 bp, Artificial sequence
  Primer
ggatggcagagtccagct

SEQ ID NO: 129 - DNA, 24 bp, Artificial sequence
  Primer
aaaaaacaagaagtgcaagaagtc

SEQ ID NO: 130 - DNA, 24 bp, Artificial sequence
  Primer
taaagtttacattttttcccacca

SEQ ID NO: 131 - DNA, 27 bp, Artificial sequence
  Primer
tagctagtgttgaattatcattatcga

SEQ ID NO: 132 - DNA, 20 bp, Artificial sequence
  Primer
atcttgagaagcactgaggc

SEQ ID NO: 133 - DNA, 24 bp, Artificial sequence
  Primer
tgtcatttctgagtgtactggaga

SEQ ID NO: 134 - DNA, 18 bp, Artificial sequence
  Primer
ctccgtggtcaggctctc

SEQ ID NO: 135 - DNA, 21 bp, Artificial sequence
  Primer
aatcgcatttcaaaaatcacc

SEQ ID NO: 136 - DNA, 23 bp, Artificial sequence
  Primer
aaagaaaatcagaagatgggaaa

SEQ ID NO: 137 - DNA, 22 bp, Artificial sequence
  Primer
ttttcccagaaccatttattga

SEQ ID NO: 138 - DNA, 26 bp, Artificial sequence
  Primer
ttaaaaattcacatgtatgttttccc

SEQ ID NO: 139 - DNA, 23 bp, Artificial sequence
  Primer
atttaaggtagatgccaggaatg

SEQ ID NO: 140 - DNA, 19 bp, Artificial sequence
  Primer
tctgagctgagcaaacagc

SEQ ID NO: 141 - DNA, 23 bp, Artificial sequence
  Primer
tgggcagttgttttattttttaa

SEQ ID NO: 142 - DNA, 21 bp, Artificial sequence
  Primer
aacagtattctggggacttgc

SEQ ID NO: 143 - DNA, 20 bp, Artificial sequence
  Primer
aaggaaccaaatgtcttggc

SEQ ID NO: 144 - DNA, 30 bp, Artificial sequence
  Primer
catgattcttttatgactagatattgaatg

SEQ ID NO: 145 - DNA, 20 bp, Artificial sequence
  Primer
tcttgtaaacagctggctgc

SEQ ID NO: 146 - DNA, 24 bp, Artificial sequence
  Primer
taacactgatggatctggtatgac

SEQ ID NO: 147 - DNA, 24 bp, Artificial sequence
  Primer
crgtatattgtmgcagttacagct

SEQ ID NO: 148 - DNA, 22 bp, Artificial sequence
  Primer
aattaaaaacaagccaatctgg

SEQ ID NO: 149 - DNA, 23 bp, Artificial sequence
  Primer
gttgttgcawagttcttctaggg

SEQ ID NO: 150 - DNA, 23 bp, Artificial sequence
  Primer
aaaaaagaaatcttccttccctt

SEQ ID NO: 151 - DNA, 27 bp, Artificial sequence
  Primer
accttcttaacttctgcctaaactatt

SEQ ID NO: 152 - DNA, 23 bp, Artificial sequence
  Primer
acgtatttgtagctgtttgtacg

SEQ ID NO: 153 - DNA, 19 bp, Artificial sequence
  Primer
agcccaacaagaaagagga

SEQ ID NO: 154 - DNA, 23 bp, Artificial sequence
  Primer
tattagaatctcagggagaggga

SEQ ID NO: 155 - DNA, 23 bp, Artificial sequence
  Primer
acacaatcccatgaacagtaaaa

SEQ ID NO: 156 - DNA, 23 bp, Artificial sequence
  Primer
actgactgtaaaatcctgagcag

SEQ ID NO: 157 - DNA, 21 bp, Artificial sequence
  Primer
acaaccctaggtcagagatgg

SEQ ID NO: 158 - DNA, 24 bp, Artificial sequence
  Primer
tttatgggaaattggttatgactt

SEQ ID NO: 159 - DNA, 19 bp, Artificial sequence
  Primer
acacagcaccagcatgatg

SEQ ID NO: 160 - DNA, 23 bp, Artificial sequence
  Primer
cacctggagatatttgattcatg

SEQ ID NO: 161 - DNA, 27 bp, Artificial sequence
  Primer
gactgtttcagtactgttttcttgttt

SEQ ID NO: 162 - DNA, 27 bp, Artificial sequence
  Primer
aagtactttgaaggatgtaatgcttat

SEQ ID NO: 163 - DNA, 27 bp, Artificial sequence
  Primer
ctctacttctatttcaatctccatcat

SEQ ID NO: 164 - DNA, 24 bp, Artificial sequence
  Primer
aatttggaaccaatttggtaatat

SEQ ID NO: 165 - DNA, 19 bp, Artificial sequence
  Primer
tttgatctcacccccttcc

SEQ ID NO: 166 - DNA, 26 bp, Artificial sequence
  Primer
acagaaagccagattgtataactttc

SEQ ID NO: 167 - DNA, 20 bp, Artificial sequence
  Primer
tttcccatatctgttgctcc

SEQ ID NO: 168 - DNA, 25 bp, Artificial sequence
  Primer
attccatactgcataacacatttct

SEQ ID NO: 169 - DNA, 26 bp, Artificial sequence
  Primer
tgaaatcatatcagtggactttttaa

SEQ ID NO: 170 - DNA, 20 bp, Artificial sequence
  Primer
attcacaaatgctctgtgcc

SEQ ID NO: 171 - DNA, 19 bp, Artificial sequence
  Primer
aacatcccatcagtggtcc

SEQ ID NO: 172 - DNA, 18 bp, Artificial sequence
  Primer
atgtccaggcctctccca

SEQ ID NO: 173 - DNA, 18 bp, Artificial sequence
  Primer
ggctgaggactgtggagc

SEQ ID NO: 174 - DNA, 27 bp, Artificial sequence
  Primer
aaaatactgttaaaaagggtcttcttg

SEQ ID NO: 175 - DNA, 20 bp, Artificial sequence
  Primer
aatgctgtgtctgggaagag

SEQ ID NO: 176 - DNA, 20 bp, Artificial sequence
  Primer
acytgatacagcgtgtggac

SEQ ID NO: 177 - DNA, 19 bp, Artificial sequence
  Primer
agcctgtgggtctgagtca

SEQ ID NO: 178 - DNA, 27 bp, Artificial sequence
  Primer
ttcttgcatatattttatttctttccc

SEQ ID NO: 179 - DNA, 20 bp, Artificial sequence
  Primer
ccaagaggcctagaatctcc

SEQ ID NO: 180 - DNA, 22 bp, Artificial sequence
  Primer
aaaacatctgaaaattgcacag

SEQ ID NO: 181 - DNA, 26 bp, Artificial sequence
  Primer
tgtaaccctctgttgtgtagtatacg

SEQ ID NO: 182 - DNA, 23 bp, Artificial sequence
  Primer
ttatattctggggacatmttgct

SEQ ID NO: 183 - DNA, 23 bp, Artificial sequence
  Primer
tctctgtcattggtaagttctgg

SEQ ID NO: 184 - DNA, 31 bp, Artificial sequence
  Primer
atggtaggaactacagaattgtatttaatat

SEQ ID NO: 185 - DNA, 21 bp, Artificial sequence
  Primer
taggacctccgtaatctcacc

SEQ ID NO: 186 - DNA, 27 bp, Artificial sequence
  Primer
atagggttgtagattagaatgaaatga

SEQ ID NO: 187 - DNA, 28 bp, Artificial sequence
  Primer
tggaaagactattaggtcataggttatt

SEQ ID NO: 188 - DNA, 27 bp, Artificial sequence
  Primer
agtgtagctagtaggtgtttgtctctc

SEQ ID NO: 189 - DNA, 21 bp, Artificial sequence
  Primer
aattcgcattgaggaaaaatg

SEQ ID NO: 190 - DNA, 22 bp, Artificial sequence
  Primer
aaaaaattacaggacatgccaa

SEQ ID NO: 191 - DNA, 18 bp, Artificial sequence
  Primer
agcaggggctttagagca

SEQ ID NO: 192 - DNA, 19 bp, Artificial sequence
  Primer
gtttgcgctattgcttctg

SEQ ID NO: 193 - DNA, 30 bp, Artificial sequence
  Primer
tacatagagtaatacttggctacatgagtt

SEQ ID NO: 194 - DNA, 29 bp, Artificial sequence
  Primer
taatacctacatttacaagaaccttcatt

SEQ ID NO: 195 - DNA, 23 bp, Artificial sequence
  Primer
ataagagttggtcaaaagtgggt

SEQ ID NO: 196 - DNA, 27 bp, Artificial sequence
  Primer
aatgaggattcctttcattattaattc

SEQ ID NO: 197 - DNA, 22 bp, Artificial sequence
  Primer
aaatgccatagtcactccaaag

SEQ ID NO: 198 - DNA, 28 bp, Artificial sequence
  Primer
ttaatagaacaaaatgaggaaaactcta

SEQ ID NO: 199 - DNA, 23 bp, Artificial sequence
  Primer
ttccaaagtaagacatgaaaacc

SEQ ID NO: 200 - DNA, 22 bp, Artificial sequence
  Primer
aacatgctttgagaatgttgtg

SEQ ID NO: 201 - DNA, 23 bp, Artificial sequence
  Primer
aaatctgacaaacattttcgtga

SEQ ID NO: 202 - DNA, 20 bp, Artificial sequence
  Primer
gggtcactcccttcttctca

SEQ ID NO: 203 - DNA, 30 bp, Artificial sequence
  Primer
tatttgtcactaagaatgagtcagtataga

SEQ ID NO: 204 - DNA, 24 bp, Artificial sequence
  Primer
aaaaaacaatagctctcagaggac

SEQ ID NO: 205 - DNA, 26 bp, Artificial sequence
  Primer
taaggaaaatccttaatcttatcagc

SEQ ID NO: 206 - DNA, 23 bp, Artificial sequence
  Primer
ttaaatgatgccatgctttactg

SEQ ID NO: 207 - DNA, 22 bp, Artificial sequence
  Primer
tcctagagaagttgagccatca

SEQ ID NO: 208 - DNA, 18 bp, Artificial sequence
  Primer
attttggctgcagaatgg

SEQ ID NO: 209 - DNA, 25 bp, Artificial sequence
  Primer
tttacttgaacaggatttggtttag

SEQ ID NO: 210 - DNA, 24 bp, Artificial sequence
  Primer
aaatgattaatgatggattttcca

SEQ ID NO: 211 - DNA, 26 bp, Artificial sequence
  Primer
aaagaaactgctttctatggtagaca

SEQ ID NO: 212 - DNA, 21 bp, Artificial sequence
  Primer
atactgtttctgaggcagctg

SEQ ID NO: 213 - DNA, 26 bp, Artificial sequence
  Primer
tcatatgctaatgacctcattttaaa

SEQ ID NO: 214 - DNA, 32 bp, Artificial sequence
  Primer
cattttttagtataagtatgttttgaagactg

SEQ ID NO: 215 - DNA, 22 bp, Artificial sequence
  Primer
acgtctttttggattgatagga

SEQ ID NO: 216 - DNA, 22 bp, Artificial sequence
  Primer
agtctcctcaccgttacttgag

SEQ ID NO: 217 - DNA, 23 bp, Artificial sequence
  Primer
tcacatgtaaaaccacaaaaaca

SEQ ID NO: 218 - DNA, 28 bp, Artificial sequence
  Primer
ttggtacatattcacatactttttttct

SEQ ID NO: 219 - DNA, 26 bp, Artificial sequence
  Primer
aaatatcctgaatgcttaaaatgaag

SEQ ID NO: 220 - DNA, 22 bp, Artificial sequence
  Primer
ttgatcactggaatgaaactca

SEQ ID NO: 221 - DNA, 18 bp, Artificial sequence
  Primer
agtcagcaagagcccaag

SEQ ID NO: 222 - DNA, 19 bp, Artificial sequence
  Primer
ttgttgttcgtgcaggttt

SEQ ID NO: 223 - DNA, 18 bp, Artificial sequence
  Primer
tgtgaatcccaggggagt

SEQ ID NO: 224 - DNA, 22 bp, Artificial sequence
  Primer
ttacgtatcaagccaaaagagg

SEQ ID NO: 225 - DNA, 29 bp, Artificial sequence
  Primer
cacagatgacaaagtattaaaattatagc

SEQ ID NO: 226 - DNA, 18 bp, Artificial sequence
  Primer
tttccctggctactggca

SEQ ID NO: 227 - DNA, 26 bp, Artificial sequence
  Primer
tagacataaattctagcagcaacatt

SEQ ID NO: 228 - DNA, 29 bp, Artificial sequence
  Primer
aaaatgtctacttttcaggtatattagga

SEQ ID NO: 229 - DNA, 24 bp, Artificial sequence
  Primer
ataaggctcacataatagtggatg

SEQ ID NO: 230 - DNA, 22 bp, Artificial sequence
  Primer
acttttcaccactcagaaggat

SEQ ID NO: 231 - DNA, 20 bp, Artificial sequence
  Primer
aggctgtcatgcttcttcat

SEQ ID NO: 232 - DNA, 32 bp, Artificial sequence
  Primer
agaatcattattaagtcattaaggtaagtatg

SEQ ID NO: 233 - DNA, 23 bp, Artificial sequence
  Primer
gccaggttagtctaatgtttcaa

SEQ ID NO: 234 - DNA, 19 bp, Artificial sequence
  Primer
atgaatcctctgccacaca

SEQ ID NO: 235 - DNA, 22 bp, Artificial sequence
  Primer
ggcagatggttctgaatttaaa

SEQ ID NO: 236 - DNA, 21 bp, Artificial sequence
  Primer
aaagacaagcaaaagggagaa

SEQ ID NO: 237 - DNA, 19 bp, Artificial sequence
  Primer
tagactctgccatgcgtca

SEQ ID NO: 238 - DNA, 20 bp, Artificial sequence
  Primer
acaccccatctcctctacct

SEQ ID NO: 239 - DNA, 25 bp, Artificial sequence
  Primer
gaattttcttccacttagaaaacct

SEQ ID NO: 240 - DNA, 24 bp, Artificial sequence
  Primer
tgactttctttttttccttagcac

SEQ ID NO: 241 - DNA, 21 bp, Artificial sequence
  Primer
ataaactgtgcgtcctgagag

SEQ ID NO: 242 - DNA, 23 bp, Artificial sequence
  Primer
csargtcacatataggaatgaag

SEQ ID NO: 243 - DNA, 30 bp, Artificial sequence
  Primer
taaacactgtgatctgatattattaaaacc

SEQ ID NO: 244 - DNA, 25 bp, Artificial sequence
  Primer
tgaatctgtgcttgtagtctttttt

SEQ ID NO: 245 - DNA, 21 bp, Artificial sequence
  Primer
aattttccagccttcaaaaca

SEQ ID NO: 246 - DNA, 28 bp, Artificial sequence
  Primer
ataagtagaagcacttcattacttagcc

SEQ ID NO: 247 - DNA, 25 bp, Artificial sequence
  Primer
acaaacaggtagaacacaagatttt

SEQ ID NO: 248 - DNA, 23 bp, Artificial sequence
  Primer
tagttgatgaggatggagtctga

SEQ ID NO: 249 - DNA, 27 bp, Artificial sequence
  Primer
tataaagtgaatgaaaaaagggagata

SEQ ID NO: 250 - DNA, 24 bp, Artificial sequence
  Primer
atagactgtagatggcctaaggac

SEQ ID NO: 251 - DNA, 18 bp, Artificial sequence
  Primer
ggggcaaagctaaatggc

SEQ ID NO: 252 - DNA, 20 bp, Artificial sequence
  Primer
acaaaagccgtgaacttgag

SEQ ID NO: 253 - DNA, 23 bp, Artificial sequence
  Primer
gcttagagagatagttgagggca

SEQ ID NO: 254 - DNA, 21 bp, Artificial sequence
  Primer
aatccacatgcctaccttagg

SEQ ID NO: 255 - DNA, 20 bp, Artificial sequence
  Primer
tcctgtacctgccttgatct

SEQ ID NO: 256 - DNA, 23 bp, Artificial sequence
  Primer
atctcacctgctttcttagatgc

SEQ ID NO: 257 - DNA, 19 bp, Artificial sequence
  Primer
gaycaccccgccttgtcta

SEQ ID NO: 258 - DNA, 26 bp, Artificial sequence
  Primer
gccttattgttttttatttcatgatc

SEQ ID NO: 259 - DNA, 25 bp, Artificial sequence
  Primer
acttcctctctgttagggataacat

SEQ ID NO: 260 - DNA, 27 bp, Artificial sequence
  Primer
taagtttgatggatttttcctactatg

SEQ ID NO: 261 - DNA, 25 bp, Artificial sequence
  Primer
atgagttcatatgagtaaagatgct

SEQ ID NO: 262 - DNA, 25 bp, Artificial sequence
  Primer
ttaacagcatcctcccctcggcaaa

SEQ ID NO: 263 - DNA, 25 bp, Artificial sequence
  Primer
catcttcattcacagggagaaaaca

SEQ ID NO: 264 - DNA, 25 bp, Artificial sequence
  Primer
gcaagggcatagtcttctttatggg

SEQ ID NO: 265 - DNA, 25 bp, Artificial sequence
  Primer
caggaacaacctcagtacatacaac

SEQ ID NO: 266 - DNA, 25 bp, Artificial sequence
  Primer
ttggtctctgctgaacagcccgaca

SEQ ID NO: 267 - DNA, 25 bp, Artificial sequence
  Primer
tcaatctatgatgaaggaggcaagc

SEQ ID NO: 268 - DNA, 25 bp, Artificial sequence
  Primer
atctcaggggacttgggggtttcgc

SEQ ID NO: 269 - DNA, 25 bp, Artificial sequence
  Primer
agaaatgctgtttttctcctgacac

SEQ ID NO: 270 - DNA, 22 bp, Artificial sequence
  Primer
agtcctaagcttgcctaccttc

SEQ ID NO: 271 - DNA, 25 bp, Artificial sequence
  Primer
aacaaagggaactgtragttgatct

SEQ ID NO: 272 - DNA, 25 bp, Artificial sequence
  Primer
cttttcaacccaagtggaaacccag

SEQ ID NO: 273 - DNA, 25 bp, Artificial sequence
  Primer
gcccccttgacagtgagacttcctt

SEQ ID NO: 274 - DNA, 25 bp, Artificial sequence
  Primer
tgtattttaaaataaattacaagca

SEQ ID NO: 275 - DNA, 25 bp, Artificial sequence
  Primer
caaatggcccgtaaagcamgtgtgc

SEQ ID NO: 276 - DNA, 25 bp, Artificial sequence
  Primer
agatagtgtgtctcttgagcactga

SEQ ID NO: 277 - DNA, 25 bp, Artificial sequence
  Primer
ccctgctctcaaaagccacgtagag

SEQ ID NO: 278 - DNA, 25 bp, Artificial sequence
  Primer
gggctgtgaagataggaccaagtat

SEQ ID NO: 279 - DNA, 25 bp, Artificial sequence
  Primer
tccctgtgtgctaaattcacatagc

SEQ ID NO: 280 - DNA, 25 bp, Artificial sequence
  Primer
acgaggggacgagaatcaggctgag

SEQ ID NO: 281 - DNA, 25 bp, Artificial sequence
  Primer
aagagagggaaatgtatcattggca

SEQ ID NO: 282 - DNA, 25 bp, Artificial sequence
  Primer
ctcgccagatattagatcaacaacc

SEQ ID NO: 283 - DNA, 25 bp, Artificial sequence
  Primer
tttcgttggccttgcgctctccatc

SEQ ID NO: 284 - DNA, 25 bp, Artificial sequence
  Primer
cygcaggtatacatgggtccattcc

SEQ ID NO: 285 - DNA, 25 bp, Artificial sequence
  Primer
caatattcrtaccagataaattcca

SEQ ID NO: 286 - DNA, 25 bp, Artificial sequence
  Primer
acggtgccctggactgcaaggtgcc

SEQ ID NO: 287 - DNA, 25 bp, Artificial sequence
  Primer
cagagccaagcctccatgaacccac

SEQ ID NO: 288 - DNA, 25 bp, Artificial sequence
  Primer
gcaacgagagaaagactcatatagt

SEQ ID NO: 289 - DNA, 25 bp, Artificial sequence
  Primer
ctcagatccatttcagtagctcatc

SEQ ID NO: 290 - DNA, 25 bp, Artificial sequence
  Primer
tccccaagcccccaaccctcactcc

SEQ ID NO: 291 - DNA, 25 bp, Artificial sequence
  Primer
ttcttgtttgcagtattgcttggtc

SEQ ID NO: 292 - DNA, 25 bp, Artificial sequence
  Primer
atgcttatctctggggaaagtatgt

SEQ ID NO: 293 - DNA, 25 bp, Artificial sequence
  Primer
ccatcatkaacatcattgatgctca

SEQ ID NO: 294 - DNA, 25 bp, Artificial sequence
  Primer
gtwgtatacatataaactcatrgat

SEQ ID NO: 295 - DNA, 25 bp, Artificial sequence
  Primer
aacaaaaaccatgtcagtcaaacat

SEQ ID NO: 296 - DNA, 25 bp, Artificial sequence
  Primer
taactttccattgatacatagatgc

SEQ ID NO: 297 - DNA, 25 bp, Artificial sequence
  Primer
aacttttactttcgagtcttgaggg

SEQ ID NO: 298 - DNA, 25 bp, Artificial sequence
  Primer
tgtatgttttcccattgcattaaat

SEQ ID NO: 299 - DNA, 25 bp, Artificial sequence
  Primer
ttttaataatgttatgttaaaatcc

SEQ ID NO: 300 - DNA, 25 bp, Artificial sequence
  Primer
ccgaagcagattcagggccctccaa

SEQ ID NO: 301 - DNA, 25 bp, Artificial sequence
  Primer
caacgggcatmcacagagaccccat

SEQ ID NO: 302 - DNA, 25 bp, Artificial sequence
  Primer
tgcggatccaaatgctcccaacagc

SEQ ID NO: 303 - DNA, 25 bp, Artificial sequence
  Primer
agatacagaagatgcaggaggaaga

SEQ ID NO: 304 - DNA, 25 bp, Artificial sequence
  Primer
atacacgatgcttccctatggtaaa

SEQ ID NO: 305 - DNA, 25 bp, Artificial sequence
  Primer
ctgacaggcaagtcctctgatcctc

SEQ ID NO: 306 - DNA, 25 bp, Artificial sequence
  Primer
acgtaggcactrtcaggggaggtac

SEQ ID NO: 307 - DNA, 25 bp, Artificial sequence
  Primer
ctcgagcccagtgacccccctcatc

SEQ ID NO: 308 - DNA, 25 bp, Artificial sequence
  Primer
tttcccagtacatactattgtgctt

SEQ ID NO: 309 - DNA, 25 bp, Artificial sequence
  Primer
cagacaagttgtcccagccctgccg

SEQ ID NO: 310 - DNA, 22 bp, Artificial sequence
  Primer
tggcaatgttcctgattgttcc

SEQ ID NO: 311 - DNA, 25 bp, Artificial sequence
  Primer
ggagttttctttttattcctgtatg

SEQ ID NO: 312 - DNA, 25 bp, Artificial sequence
  Primer
atctggtccacaatccagacagttc

SEQ ID NO: 313 - DNA, 25 bp, Artificial sequence
  Primer
gttggcatgacaaggatctgggtca

SEQ ID NO: 314 - DNA, 25 bp, Artificial sequence
  Primer
cactgttcaccragcaaacggaatg

SEQ ID NO: 315 - DNA, 25 bp, Artificial sequence
  Primer
tgaatgggaagtgggtgtgatggaa

SEQ ID NO: 316 - DNA, 25 bp, Artificial sequence
  Primer
accctctttrtgtgccagattaagt

SEQ ID NO: 317 - DNA, 25 bp, Artificial sequence
  Primer
gccagaagaaaaatgtatgcaatat

SEQ ID NO: 318 - DNA, 25 bp, Artificial sequence
  Primer
atctcaaattggaagaaggttttta

SEQ ID NO: 319 - DNA, 25 bp, Artificial sequence
  Primer
gatctggaatgtggtagtgaytagt

SEQ ID NO: 320 - DNA, 25 bp, Artificial sequence
  Primer
aaygtccayrttgttcgagaatctc

SEQ ID NO: 321 - DNA, 25 bp, Artificial sequence
  Primer
ctggtccaggaagatcccacatgcc

SEQ ID NO: 322 - DNA, 25 bp, Artificial sequence
  Primer
taaattgagaggaaatgatraagtg

SEQ ID NO: 323 - DNA, 25 bp, Artificial sequence
  Primer
gattaaccctcaaaaactgaaagca

SEQ ID NO: 324 - DNA, 25 bp, Artificial sequence
  Primer
ttctcacaacatgcttgtctttact

SEQ ID NO: 325 - DNA, 25 bp, Artificial sequence
  Primer
agtgtgtgtgtgtgaaatcagcaga

SEQ ID NO: 326 - DNA, 25 bp, Artificial sequence
  Primer
caagcattattttaaacaggcaaac

SEQ ID NO: 327 - DNA, 25 bp, Artificial sequence
  Primer
tttttcaactttgcaaaagtaaaac

SEQ ID NO: 328 - DNA, 25 bp, Artificial sequence
  Primer
caatattggacaatttgttagtagc

SEQ ID NO: 329 - DNA, 25 bp, Artificial sequence
  Primer
ataagtgaacagggggtttggtgtg

SEQ ID NO: 330 - DNA, 25 bp, Artificial sequence
  Primer
acggatgagattcatttgaactggc

SEQ ID NO: 331 - DNA, 25 bp, Artificial sequence
  Primer
agtgatttttctggagccagactgc

SEQ ID NO: 332 - DNA, 25 bp, Artificial sequence
  Primer
ccagcrtgagaaatactgacrgtga

SEQ ID NO: 333 - DNA, 25 bp, Artificial sequence
  Primer
ttatctcccaaaagatagagcttca

SEQ ID NO: 334 - DNA, 25 bp, Artificial sequence
  Primer
ttgccttgcttcgtttgttatgagt

SEQ ID NO: 335 - DNA, 25 bp, Artificial sequence
  Primer
aatagattattttctggagaatacc

SEQ ID NO: 336 - DNA, 25 bp, Artificial sequence
  Primer
gatcagaggagacaatgtctgttgg

SEQ ID NO: 337 - DNA, 25 bp, Artificial sequence
  Primer
gttaccagtttagggaacctaccac

SEQ ID NO: 338 - DNA, 22 bp, Artificial sequence
  Primer
agtgaatttgtacaaggcttcc

SEQ ID NO: 339 - DNA, 25 bp, Artificial sequence
  Primer
taagaacccagacatttttacaaac

SEQ ID NO: 340 - DNA, 25 bp, Artificial sequence
  Primer
aatattgatcttgtgtagtatatgc

SEQ ID NO: 341 - DNA, 25 bp, Artificial sequence
  Primer
tgtctggggaactgaggtcagccgc

SEQ ID NO: 342 - DNA, 25 bp, Artificial sequence
  Primer
agcagtgagtttactttatggaata

SEQ ID NO: 343 - DNA, 25 bp, Artificial sequence
  Primer
acttcagaatgtggccatatttgga

SEQ ID NO: 344 - DNA, 25 bp, Artificial sequence
  Primer
agaaaaggaaaacacatacacacac

SEQ ID NO: 345 - DNA, 25 bp, Artificial sequence
  Primer
atttaaacatttccttttagattga

SEQ ID NO: 346 - DNA, 25 bp, Artificial sequence
  Primer
cagcctaggttgagacattcagcag

SEQ ID NO: 347 - DNA, 25 bp, Artificial sequence
  Primer
tttagtgtataacagttaaaaatga

SEQ ID NO: 348 - DNA, 25 bp, Artificial sequence
  Primer
catccttcagaaaaatgccagtgac

SEQ ID NO: 349 - DNA, 25 bp, Artificial sequence
  Primer
atctgcaatttaaaatggtggcatg

SEQ ID NO: 350 - DNA, 25 bp, Artificial sequence
  Primer
cttccaactgagcaaataaagtttc

SEQ ID NO: 351 - DNA, 25 bp, Artificial sequence
  Primer
cgtttctctggaatttcctattctt

SEQ ID NO: 352 - DNA, 25 bp, Artificial sequence
  Primer
aatcgagaaatgaaaataatggagg

SEQ ID NO: 353 - DNA, 25 bp, Artificial sequence
  Primer
attatgatctatcagaatgatttac

SEQ ID NO: 354 - DNA, 25 bp, Artificial sequence
  Primer
cttcacaaataaaattcactcaatc

SEQ ID NO: 355 - DNA, 25 bp, Artificial sequence
  Primer
attgtgatttttcaaatgtttgtca

SEQ ID NO: 356 - DNA, 25 bp, Artificial sequence
  Primer
gtagatcaaaggaagtgcagatgcc

SEQ ID NO: 357 - DNA, 25 bp, Artificial sequence
  Primer
agagaaacraagtgctgttttcaat

SEQ ID NO: 358 - DNA, 25 bp, Artificial sequence
  Primer
cactgaaatgaaacctctaaataca

SEQ ID NO: 359 - DNA, 25 bp, Artificial sequence
  Primer
agatgaaagtgaatgataagcattt

SEQ ID NO: 360 - DNA, 25 bp, Artificial sequence
  Primer
agaggacatggaggcagaatgcaga

SEQ ID NO: 361 - DNA, 25 bp, Artificial sequence
  Primer
gtcttggctccctatgaccgtgtca

SEQ ID NO: 362 - DNA, 25 bp, Artificial sequence
  Primer
ttcttgaagattctgttaccattat

SEQ ID NO: 363 - DNA, 25 bp, Artificial sequence
  Primer
tttatttgtaagcatggtgaattac

SEQ ID NO: 364 - DNA, 25 bp, Artificial sequence
  Primer
ttcgtgggttgttcttcctgtttgc

SEQ ID NO: 365 - DNA, 25 bp, Artificial sequence
  Primer
tcagtacataaacagagtcattgcc

SEQ ID NO: 366 - DNA, 25 bp, Artificial sequence
  Primer
gtgttaacaacatttgcatctctga

SEQ ID NO: 367 - DNA, 25 bp, Artificial sequence
  Primer
agcaagtcagtctgtggaggcggca

SEQ ID NO: 368 - DNA, 25 bp, Artificial sequence
  Primer
tacagcaactattattcaatctttt

SEQ ID NO: 369 - DNA, 25 bp, Artificial sequence
  Primer
gagagataggttcataagcttgttg

SEQ ID NO: 370 - DNA, 25 bp, Artificial sequence
  Primer
gccctcctgcaagttaggttcttta

SEQ ID NO: 371 - DNA, 25 bp, Artificial sequence
  Primer
acaaggtgtgccctgaaataagaac

SEQ ID NO: 372 - DNA, 25 bp, Artificial sequence
  Primer
ttaaagtgctgaaaacgaaagctgg

SEQ ID NO: 373 - DNA, 25 bp, Artificial sequence
  Primer
tatttggaaaattctgatacaaaga

SEQ ID NO: 374 - DNA, 25 bp, Artificial sequence
  Primer
tgaagacccagcactgccagaaata

SEQ ID NO: 375 - DNA, 25 bp, Artificial sequence
  Primer
gactcttagtccagacttttctgac

SEQ ID NO: 376 - DNA, 25 bp, Artificial sequence
  Primer
agctggttttattttccttctttcc

SEQ ID NO: 377 - DNA, 25 bp, Artificial sequence
  Primer
tgacatatatccatcaatataatac

SEQ ID NO: 378 - DNA, 25 bp, Artificial sequence
  Primer
gtaatctcaggcagggcgggtaatg

SEQ ID NO: 379 - DNA, 25 bp, Artificial sequence
  Primer
cattccaatggcatcaaatgcctcc

SEQ ID NO: 380 - DNA, 25 bp, Artificial sequence
  Primer
tcttacccactcttccatcagcacc

SEQ ID NO: 381 - DNA, 25 bp, Artificial sequence
  Primer
catctgcacagtaagaacagcgagc

SEQ ID NO: 382 - DNA, 25 bp, Artificial sequence
  Primer
gacatgagaaagataaagacctcaa

SEQ ID NO: 383 - DNA, 25 bp, Artificial sequence
  Primer
ccagtccatctccaccaggagccca

SEQ ID NO: 384 - DNA, 25 bp, Artificial sequence
  Primer
cccaggccacagtgtccatgtaccc

SEQ ID NO: 385 - DNA, 25 bp, Artificial sequence
  Primer
cctcttcccatccaatctacataac

SEQ ID NO: 386 - DNA, 25 bp, Artificial sequence
  Primer
cggattttcaagactcccctacgcc

SEQ ID NO: 387 - DNA, 25 bp, Artificial sequence
  Primer
ggagctggaggaggtgcaagacgac

SEQ ID NO: 388 - DNA, 25 bp, Artificial sequence
  Primer
gggcgagagtgcaggagctcagggc

SEQ ID NO: 389 - DNA, 25 bp, Artificial sequence
  Primer
ctttcctccccacaaaagaacctaa

SEQ ID NO: 390 - DNA, 25 bp, Artificial sequence
  Primer
cctaatttagcttgaaaatgagttc